(19) United States
(12) Patent Application Publication
(10) Pub. No.: US 2025/0114033 A1
(43) Pub. Date: Apr. 10, 2025

(54) DETECTION SYSTEM

(71) Applicants: Japan Display Inc., Tokyo (JP); National University Corporation Tokyo Medical and Dental University, Tokyo (JP)

(72) Inventors: Yuji ONO, Tokyo (JP); Kei TAMURA, Tokyo (JP); Hiromi MITANI, Toyko (JP); Hiroko OHMORI, Tokyo (JP); Takashi ONO, Tokyo (JP); Hiroaki KIRIMOTO, Tokyo (JP)

(21) Appl. No.: 18/905,730

(22) Filed: Oct. 3, 2024

(30) Foreign Application Priority Data
Oct. 4, 2023 (JP) 2023-172698

(51) Int. Cl. A61B 5/00; A61B 5/389

(52) U.S. Cl. CPC A61B 5/4557; A61B 5/389; A61B 5/682; A61B 5/7225; A61B 2562/0247

(57) ABSTRACT

According to an aspect, a detection system includes: a detection device configured to perform sensing related to bruxism; and an information processing device configured to determine presence or absence of bruxism based on data indicating a result of sensing by the detection device. The detection device includes a force sensor disposed at a mouthpiece, a myoelectric sensor attachable to a human cheek, and a controller configured to output data in which an output of the force sensor is synchronized with an output of the myoelectric sensor.

### CROSS-REFERENCE TO RELATED APPLICATION

This application claims the benefit of priority from Japanese Patent Application No. 2023-172698 filed on Oct. 4, 2023, the entire contents of which are incorporated herein by reference.

### BACKGROUND

### 1. Technical Field

What is disclosed herein relates to a detection system.

### 2. Description of the Related Art

Methods for detecting human bruxism are known, such as a method using a mouthpiece with a force sensor (refer to, for example, Japanese Patent No. 6634567) and a method using a myoelectric sensor (refer to, for example, Japanese Patent No. 6618482).

Both the method only using a force sensor and the method only using a myoelectric sensor may fail to distinguish whether data acquired from the sensor is data caused by bruxism or data not caused by bruxism, in some cases. For example, with the method using the force sensor, it is difficult to distinguish between force caused by bruxism and force caused by tongue or lips touching the force sensor. The myoelectric sensor sometimes produces an output similar to that caused by bruxism when the eyelids are tightly closed.

For the foregoing reasons, there is a need for a detection system that can more accurately distinguish between data caused by bruxism and data not caused by bruxism.

### SUMMARY

According to an aspect, a detection system includes: a detection device configured to perform sensing related to bruxism; and an information processing device configured to determine presence or absence of bruxism based on data indicating a result of sensing by the detection device. The detection device includes a force sensor disposed at a mouthpiece, a myoelectric sensor attachable to a human cheek, and a controller configured to output data in which an output of the force sensor is synchronized with an output of the myoelectric sensor.

### BRIEF DESCRIPTION OF THE DRAWINGS

FIG. 1 is a block diagram illustrating a main configuration of a detection system having a detection device;

FIG. 2 is a diagram illustrating an exemplary outer shape of the detection device;

FIG. 3 is a schematic exploded perspective view of a main part configuration example of a first section;

FIG. 4 is a schematic side view illustrating a mechanism of a first part and a second part bonded together;

FIG. 5 is a schematic plan view of a main part configuration example of the first section;

FIG. 6 is a schematic diagram illustrating an example of attachment of the detection device to a human;

FIG. 7 is a schematic diagram illustrating the arrangement of the first section in the mouth illustrated in FIG. 6;

FIG. 8 is a schematic diagram illustrating an attachment position of first, second, and third electrodes;

FIG. 9 is a graph illustrating an example of the relation in time sequence between human bite force indicated by output of a force sensor and myoelectricity indicated by output of a myoelectric sensor;

FIG. 10 is a table illustrating first, second, and third patterns as examples of output produced in the absence of bruxism;

FIG. 11 is a flowchart illustrating an example of a process related to operation of the detection device;

FIG. 12 is a flowchart illustrating an example of a process related to operation of the detection device, which is different from that of FIG. 11;

FIG. 13 is a flowchart illustrating an example of a process related to operation of the detection device, which is different from those of FIG. 11 and FIG. 12;

FIG. 14 is a schematic diagram illustrating an example of training data;

FIG. 15 is a schematic diagram illustrating an example of feature data;

FIG. 16 is a two-dimensional graph schematically illustrating machine learning related to classification of the presence or absence of bruxism;

FIG. 17 is a two-dimensional graph schematically illustrating machine learning related to classification of types of bruxism by a subject;

FIG. 18 is a two-dimensional graph schematically illustrating machine learning related to classification of types of bruxism by a subject different from the subject in FIG. 17;

FIG. 19 is a diagram illustrating an example of the relation between distinction target data and distinction information indicating output of a distinction processor based on the distinction target data;

FIG. 20 is a flowchart illustrating an example of a process related to generation of model data; and

FIG. 21 is a flowchart illustrating a process related to estimation of occurrence of bruxism and the like based on the distinction target data.

### DETAILED DESCRIPTION

Embodiments of the present disclosure will be described below with reference to the drawings. The disclosure is only an example, and any modification that can be easily conceived by a person skilled in the art without departing from the spirit of the invention should be included in the scope of the present disclosure. In the drawings, the width, thickness, shape, and the like of each part are schematically illustrated compared with the actual manner, for the sake of clarity of explanation, but these are only by way of example and are not intended to limit the interpretation of the present disclosure. In the present description and drawings, elements similar to those illustrated in the previous drawings may be denoted with the same signs and a detailed description thereof may be omitted as appropriate.

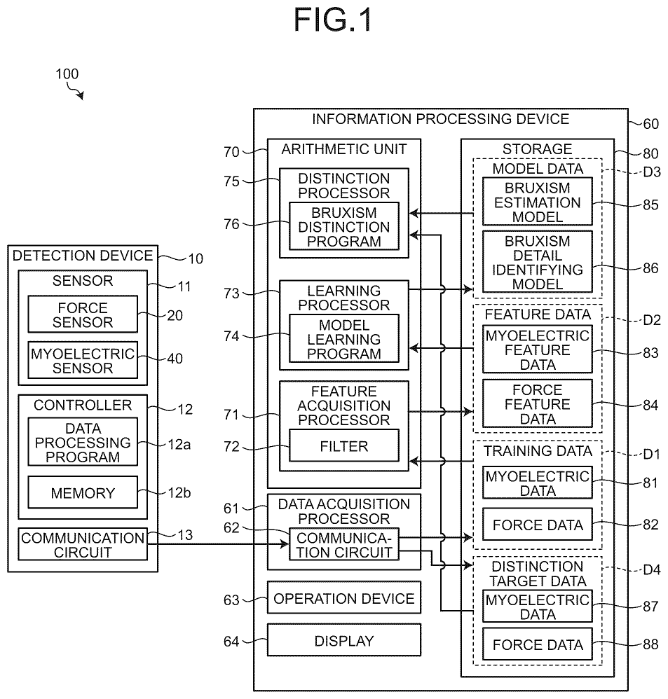

FIG. 1 is a block diagram illustrating a main configuration of a detection system 100 having a detection device 10. The detection system 100 includes the detection device 10 and an information processing device 60.

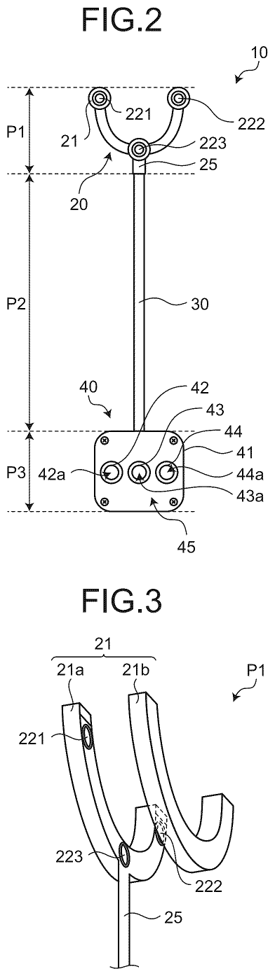

FIG. 2 is a diagram illustrating an exemplary outer shape of the detection device 10. As illustrated in FIG. 2, the detection device 10 has a first section P1, a second section P2, and a third section P3. The first section P1 includes a mouthpiece 21, a first sensor element 221, a second sensor element 222, a third sensor element 223, an FPC 25, etc. A more detailed configuration of the first section P1 will be described later in a description pertaining to a force sensor 20. The second section P2 includes an FPC 30. The third section P3 includes a casing 41, a first electrode 42, a second electrode 43, and a third electrode 44. The material of the first electrode 42, the second electrode 43, and the third electrode 44 is, for example, silver-silver chloride (Ag—AgCl) but may be gold, platinum, silver, carbon, etc.

The detection device 10 illustrated in FIG. 1 includes a sensor 11, a controller 12, and a communication circuit 13. The sensor 11 includes the force sensor 20 and a myoelectric sensor 40. The force sensor 20 and the myoelectric sensor 40 will be described sequentially below. First, the force sensor 20 will be described with reference to FIG. 3 to FIG. 7.

FIG. 3 is a schematic exploded perspective view of a main part configuration example of the first section P1. The mouthpiece 21 is formed by bonding a first part 21a and a second part 21b together. The first sensor element 221, the second sensor element 222, the third sensor element 223, and the FPC 25 are sandwiched between the first part 21a and the second part 21b.

The first sensor element 221, the second sensor element 222, and the third sensor element 223 are configured to produce an electrical effect corresponding to force. To take a specific example, the first sensor element 221, the second sensor element 222, and the third sensor element 223 are piezoelectric sensors. To take a more specific example, for example, thin-plate ceramic elements that generate a piezoelectric effect are disposed as the first sensor element 221, the second sensor element 222, and the third sensor element 223. The first sensor element 221, the second sensor element 222, and the third sensor element 223 each individually generate an electric charge corresponding to the force applied thereto.

The FPC 25 is a substrate in the form of a flexible thin film and has wiring. To take a specific example, the FPC 25 is, for example, a flexible printed circuit (FPC) board but may have another configuration that functions similarly. The first sensor element 221, the second sensor element 222, and the third sensor element 223 are wiring lines formed on the FPC 25 and are coupled to individual wire lines.

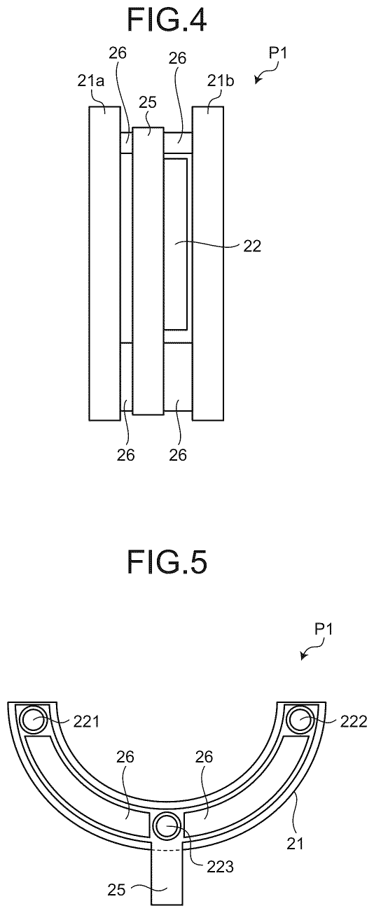

FIG. 4 is a schematic side view illustrating a mechanism of the first part 21a and the second part 21b bonded together. A sensor element 22 illustrated in FIG. 4 is any one of the first sensor element 221, the second sensor element 222, and the third sensor element 223. Hereafter, the expression “sensor element 22” refers to the first sensor element 221, the second sensor element 222, and the third sensor element 223 collectively.

As illustrated in FIG. 4, the FPC 25 is bonded to the first part 21a on one side and to the second part 21b on the other side with adhesive 26 interposed therebetween. The sensor element 22 is provided on the other side of the FPC 25 in the example illustrated in FIG. 4, but may be provided on the one side of the FPC 25.

The mouthpiece 21 is a mouthpiece adapted to a curved shape of a bifurcated portion of the FPC 25 where the first sensor element 221, the second sensor element 222, and the third sensor element 223 are provided. As illustrated in FIG. 4, the FPC 25 is adhesively fixed such that a gap is formed between the FPC 25 and the mouthpiece 21, inside the mouthpiece 21.

The mouthpiece 21 is formed, for example, but not limited to, using a resin (for example, resin or silicone) having a certain degree of flexibility. The specific composition of the mouthpiece 21 can be changed as appropriate as long as force can be transmitted to the first sensor element 221, the second sensor element 222, and the third sensor element 223. It is preferable that the mouthpiece 21 is produced by molding a dental impression acquired from each wearer.

FIG. 5 is a schematic plan view of a main part configuration example of the first section P1. As illustrated in FIGS. 2, 3, 5, and 7, the FPC 25 has a bifurcated shape at one end side having the first sensor element 221 and the second sensor element 222. The first sensor element 221 and the second sensor element 222 are provided near distal ends of the first section P1. The FPC 25 also has a strip-like shape at a proximal end side of the first section P1 that is continuously connected with the FPC 30 of the second section P2. The third sensor element 223 is provided near a connection position between the bifurcated portion and the strip portion.

As illustrated in FIG. 5, the adhesive 26 is mostly applied to, for example, but not limited to, a region between the third sensor element 223 and the first sensor element 221 and a region between the third sensor element 223 and the second sensor element 222 in the bifurcated portion. However, the adhesive may be applied in any way that can maintain the integration of the mouthpiece 21 and the FPC 25.

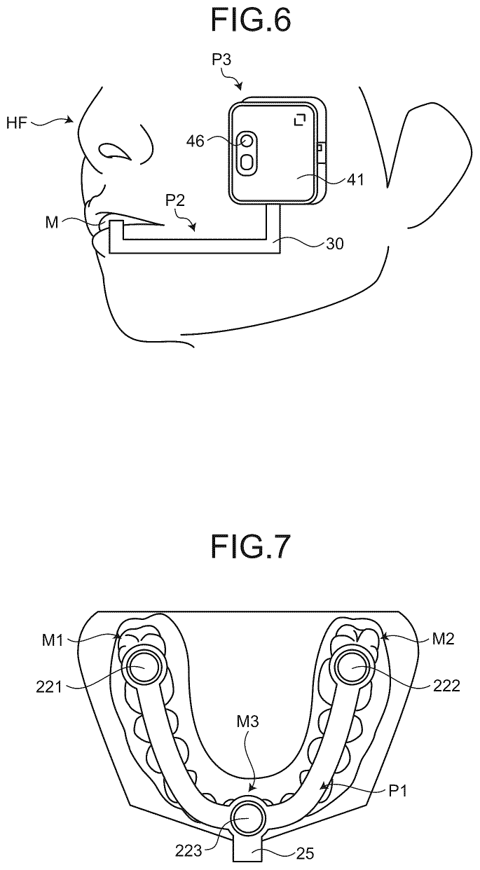

FIG. 6 is a schematic diagram illustrating an example of attachment of the detection device 10 to a human. As illustrated in FIG. 6, when the detection device 10 is attached to a human, the casing 41 of the third section P3 is attached such that a surface exposing the first electrode 42, the second electrode 43, and the third electrode 44 is in close proximity to or contact with an outer side area of the cheek of the human HF.

Specifically, for example, conductive gels 42a, 43a, and 44a are provided on the first electrode 42, the second electrode 43, and the third electrode 44 of the casing 41, respectively, and double-sided tape 45 for skin is provided on the remaining portion of the casing 41 to allow the casing 41 to be attached to the human HF. The material of the double-sided tape is preferably silicone.

In the embodiment, the double-sided tape 45 for skin is applied on the surface of the casing 41 on which the first electrode 42, the second electrode 43, and the third electrode 44 are provided. The double-sided tape 45 for skin is applied throughout the surface of the casing 41 in the area surrounding the first electrode 42, the second electrode 43, and the third electrode 44. The conductive gels 42a, 43a, and 44a cover respective outer electrode surfaces of the first electrode 42, the second electrode 43, and the third electrode 44. When the detection device 10 is attached to a human, the first section P1 is placed in the mouth M of the human. The FPC 30 of the second section P2 extends having a length by which the first section P1 can be coupled to the casing 41 when the detection device 10 is attached to the human.

The FPC 30, which is provided in the second section P2, is an FPC continuous from the proximal end side of the first section P1 of the FPC 25. The FPC 30 serves as wiring that couples the first sensor element 221, the second sensor element 222, and the third sensor element 223 to a circuit in the casing 41 of the third section P3. A circuit or the like is provided in the casing 41 to convert outputs of the first sensor element 221, the second sensor element 222, and the third sensor element 223 into digital data that can be handled individually. The circuit or the like is included in the configuration of the force sensor 20 in the embodiment.

FIG. 7 is a schematic diagram illustrating the arrangement of the first section P1 in the mouth M illustrated in FIG. 6. As illustrated in FIG. 7, the first section P1 is held in the mouth of the human such that the first sensor element 221 is located near one rearmost molar M1 among the teeth in the mouth M, the second sensor element 222 is located near the other rearmost molar M2 among the teeth in the mouth M, and the third sensor element 223 is located near incisors M3 closest to the center among the teeth in the mouth M. In other words, the shape and the extension length of the bifurcated mouthpiece 21 and the FPC 25 as well as the arrangement of the first sensor element 221, the second sensor element 222, and the third sensor element 223 in the first section P1 are predetermined such that the above positional relation between the molars M1, M2, and incisors M3 and the first, second, and third sensor elements 221, 222, and 223 is satisfied. Since there can be individual differences in the size of the human mouth M and the positions of the molars M1, M2 and incisors M3 in the mouth M, the first section P1 may be customized for each human, or a design of the first section P1 based on a relation with a typical human adult dental model may be adopted as a standard design. The first section P1 is for the upper jaw and is attached to teeth in the upper jaw. However, the first section P1 may be for the lower jaw and attached to teeth in the lower jaw. Strictly speaking, whether the first section P1 is for the upper jaw or for the lower jaw is determined for each individual human, based on the dentist's judgment. In this way, the mouthpiece 21 may be attached to either the upper jaw or the lower jaw inside the human mouth M.

When the upper and lower teeth of the jaws move to make contact at the position of the molar M1, the force caused by the contact is detected by the first sensor element 221. When the upper and lower teeth of the jaws move to make contact at the position of the molar M2, the force caused by the contact is detected by the second sensor element 222. When the upper and lower teeth of the jaws move to make contact at the position of the incisors M3, the force caused by the contact is detected by the third sensor element 223. In the embodiment, therefore, the first sensor element 221, the second sensor element 222, and the third sensor element 223 function as the force sensor 20 in the human mouth.

Either the shape of the mouthpiece 21 and the FPC 25 illustrated in FIGS. 2 and 7 or the shape of the mouthpiece 21 and the FPC 25 illustrated in FIGS. 3 to 5 may be employed. The shapes of the components of the first section P1 illustrated in FIGS. 3 to 5 are schematic shapes to illustrate the relative positional relation between the components of the first section P1 and do not represent a specific manner. The specific configuration of the first section P1 is not limited to the configuration illustrated in FIGS. 2 and 7 as long as the mouthpiece allows a force sensor to be disposed to detect force corresponding to the bite of the teeth in the human mouth. Although a case where three sensor elements 22 function as the force sensor 20 is illustrated here, a force sensor corresponding to each of teeth may be disposed. For example, if the upper jaw has 14 teeth, 14 force sensors may be disposed.

The following describes the myoelectric sensor 40 illustrated in FIG. 1 with reference to FIGS. 1, 2, 6, and 8.

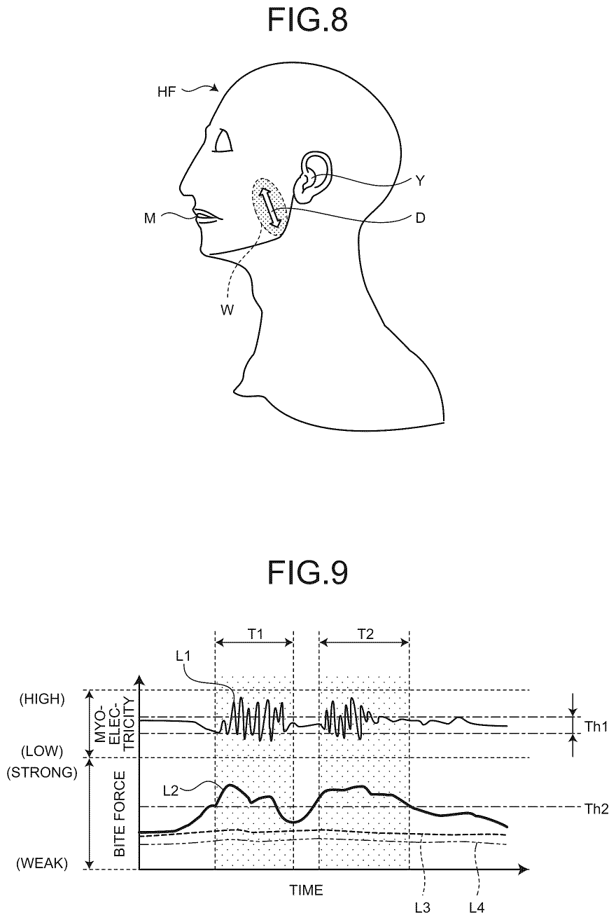

FIG. 8 is a schematic diagram illustrating an attachment position of the first electrode 42, the second electrode 43, and the third electrode 44. The casing 41 of the third section P3 is attached to the human HF such that the first electrode 42, the second electrode 43, and the third electrode 44 are aligned along a direction D in a region W illustrated in FIG. 8. The region W is a region closer to the ear Y in the human cheek area between the mouth M and the ear Y of the human HF. The direction D is, for example, a direction generally orthogonal to a straight line connecting the ear Y and the mouth M, and substantially coincides with a straight line from around the corner of one of the two eyes to the neck in the human HF. For example, as described with reference to FIG. 6 above, the casing 41 of the third section P3 is attached to the human HF such that the surface exposing the first electrode 42, the second electrode 43, and the third electrode 44 is in close proximity to or contact with the cheek area of the human HF, whereby the first electrode 42, the second electrode 43, and the third electrode 44 are aligned along the direction D in the region W.

The feature of the region W and the direction D described above are only a typical example and in practice may be adjusted for each individual human. It is preferable that the adjustment is made assuming that the positions of the first electrode 42, the second electrode 43, and the third electrode 44 in the third section P3 overlap a superficial part (superficial layer) of the human masseter muscle.

The myoelectric sensor 40 illustrated in FIG. 1 includes a function as a differential amplifier using the first electrode 42, the second electrode 43, and the third electrode 44. One of the first electrode 42, the second electrode 43, and the third electrode 44 is a reference electrode (REF). One of the first electrode 42, the second electrode 43, and the third electrode 44 that is not a reference electrode is an exploring electrode. One of the first electrode 42, the second electrode 43, and the third electrode 44 that is neither a reference electrode nor an exploring electrode is a ground electrode (GND). The ground electrode assumes a ground potential (0 V). Ideally, it is preferable that the reference electrode assumes the same potential (0 V) as the ground potential, but in practice, the reference electrode assumes a potential subjected to noise components (noise) at the position where the third section P3 is disposed. The exploring electrode is disposed in the region W to capture myoelectricity and assumes a potential corresponding to the myoelectricity. The myoelectricity refers to an electrical signal generated in the muscle corresponding to the superficial part (superficial layer) of the human masseter muscle described above.

In practice, the potential of the exploring electrode may include the noise described above. In the embodiment, therefore, similar noise is detected at the reference electrode so that a potential corresponding to myoelectricity can be detected more accurately based on the difference between the potential of the exploring electrode and the potential of the reference electrode. More specifically, an electrical signal corresponding to the difference between the potential of the exploring electrode and the potential of the ground electrode is input to one of two inputs of an amplifier including an operational amplifier or the like. An electrical signal corresponding to the difference between the potential of the reference electrode and the potential of the ground electrode is input to the other of two inputs of the amplifier. As a result, electrical signals in phase of the two inputs of the amplifier cancel each other, whereas signals different from each other, such as signals of opposite phases, are amplified. The myoelectric sensor 40 in the embodiment therefore can capture myoelectricity more accurately.

A circuit or the like is provided in the casing 41 to convert an output of the myoelectric sensor 40 serving as the amplifier described above into digital data. The circuit or the like is included in the configuration of the myoelectric sensor 40 in the embodiment.

The controller 12 illustrated in FIG. 1 performs processing based on a signal output from the sensor 11. The following describes the processing performed by the controller 12 with reference to FIG. 1 and FIGS. 9 to 13.

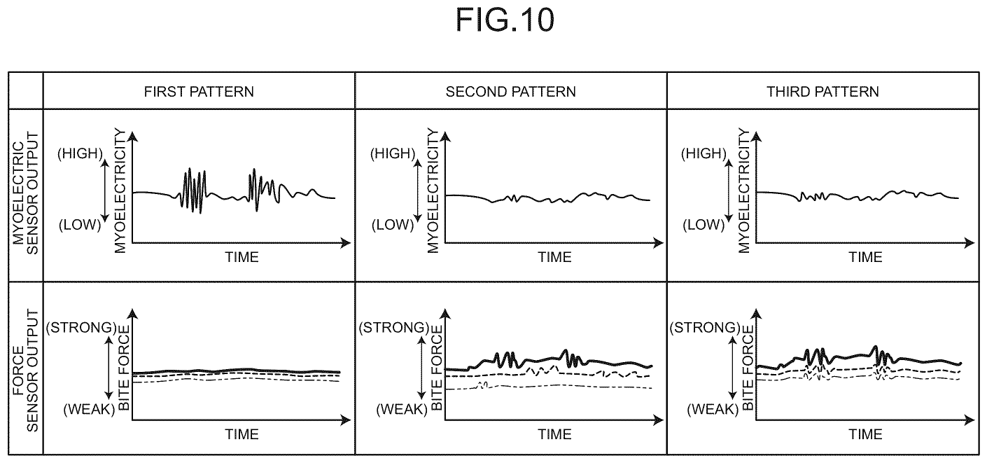

FIG. 9 is a graph illustrating an example of the relation in time sequence between human bite force indicated by output of the force sensor 20 and myoelectricity indicated by output of the myoelectric sensor 40. Among four lines in the graph illustrated in FIG. 9, lower three lines L2, L3, and L4 represent the strength of output of the force sensor 20, that is, the strength of the human bite force. The lower three lines L2, L3, and L4 represent individual outputs of the first sensor element 221, the second sensor element 222, and the third sensor element 223, respectively. Line L2 represents the output of the first sensor element 221, line L3 represents the output of the second sensor element 222, and line L4 represents the output of the third sensor element 223. Among the four lines, the upper one line L1 represents the strength of output of the myoelectric sensor 40, that is, the strength of myoelectricity. In FIG. 9, a dashed line between the lower three lines L2, L3, L4 and the upper one line L1 separates the graph representing bite force from the graph representing myoelectricity. The vertical axis direction of the graphs in FIG. 9 and FIG. 10 described later indicates the strength of output. The horizontal axis direction of the graphs in FIGS. 9 and 10 indicates the passage of time.

During periods T1 and T2 in the graph illustrated in FIG. 9, one (line L2) of the lower three lines L2, L3, and L4, which represent the strength of bite force, represents a significantly strong bite force, compared with the other two (lines L3 and L4). This indicates occurrence of a situation in which the upper and lower teeth of the jaws are in close proximity to contact each other to apply pressing force to the sensor element 22 at a position of one of molar M1, molar M2, and incisors M3. Assuming that line L2 represents output of the first sensor element 221, the situation occurs where the upper and lower teeth of the jaws are so close that they are in contact with each other at the molar M1 to apply pressing force to the sensor element 22. Such a situation can occur when bruxism is occurring. The upper one line L1 representing the strength of myoelectricity also has significantly large fluctuations of output during periods T1 and T2, compared with the other periods. This suggests movement of the masseter muscle, that is, force acting in a direction of pulling the lower jaw toward the upper jaw. This serves as a more reliably suggestion supporting the occurrence of bruxism.

As described above, in the embodiment, the occurrence of bruxism can be determined, based on data in which the output of the force sensor 20 and the output of the myoelectric sensor 40 are synchronized in time. In contrast, with only one of the output of the force sensor 20 and the output of the myoelectric sensor 40, it is difficult to accurately determine the occurrence of bruxism. The following describes a reference example in which false detection is caused with only one of the output of the force sensor 20 and the output of the myoelectric sensor 40 with reference to FIG. 10.

FIG. 10 is a table illustrating first, second, and third patterns as examples of output produced in the absence of bruxism. The first pattern is a pattern in a case where the human eyelids are tightly closed. The second pattern is a pattern in a case where the human tongue touches the teeth in the mouth. The third pattern is a pattern in a case where the human lips touch the teeth. The “myoelectric sensor output” in FIG. 10 indicates the strength of output of a configuration similar to the myoelectric sensor 40. The “force sensor output” in FIG. 10 indicates output of a configuration similar to the force sensor 20 having the first sensor element 221, the second sensor element 222, and the third sensor element 223. In the reference example, either “myoelectric sensor output” or “force sensor output” can be obtained.

As indicated by “First Pattern” in FIG. 10, the “myoelectric sensor output” has significantly large fluctuations of output even when the human eyelids are tightly closed, in the same manner as when bruxism occurs. In the reference example in which only “myoelectric sensor output” is obtained, therefore, it is difficult to distinguish between a case where bruxism occurs and a case where the human eyelids are tightly closed.

As indicated by “Second Pattern” and “Third Pattern” in FIG. 10, the output of the force sensor 20 significantly fluctuates even when the tongue or lips touch the teeth. In the reference example in which only “force sensor output” is obtained, therefore, it is difficult to distinguish between a case where bruxism occurs and a case where the tongue or lips touch the teeth.

In contrast, in the embodiment, the output of the force sensor 20 does not significantly change even when a situation similar to “First Pattern” occurs. As a result, in the embodiment, it is possible to distinguish between a case where bruxism occurs and a case where the human eyelids are tightly closed. In the embodiment, the output of the myoelectric sensor 40 does not significantly change even when a situation similar to “Second Pattern” or “Third Pattern” occurs. As a result, in the embodiment, it is possible to distinguish between a case where bruxism occurs and a case where the tongue or lips touch the teeth. The embodiment therefore can more accurately distinguish between bruxism and an event other than bruxism.

As indicated in periods T1 and T2 in FIG. 9, synchronization in time between the output of the force sensor 20 and the output of the myoelectric sensor 40 is important for distinguishing bruxism from the others. In the embodiment, the controller 12 of the detection device 10 illustrated in FIG. 1 generates data in which the output of the force sensor 20 and the output of the myoelectric sensor 40 are synchronized in time.

In the embodiment, the force sensor 20 produces an output at 8 cycles per second (8 Hz), and the myoelectric sensor 40 produces an output at 512 cycles per second (512 Hz). The controller 12 acquires the output of the force sensor 20 and the output of the myoelectric sensor 40, and generates data that can be handled on a unit time basis.

As illustrated in FIG. 1, the controller 12 includes a data processing program 12a and a memory 12b. The data processing program 12a is a software program for generating data that can be handled on a unit time basis, based on the output of the force sensor 20 and the output of the myoelectric sensor 40. The configuration of the controller 12 includes an arithmetic circuit for reading and executing the data processing program 12a. The memory 12b functions as a storage region capable of temporarily storing data representing the output of the force sensor 20 and the output of the myoelectric sensor 40 as well as data generated using the data processing program 12a. The controller 12 may be a circuit that implements the function as the data processing program 12a in hardware and has a storage region to function as the memory 12b.

The communication circuit 13 illustrated in FIG. 1 performs processing related to communication with an external apparatus such as the information processing device 60. Specifically, the communication circuit 13 has a circuit or the like to function as a network interface controller (NIC). The communication circuit 13 communicates with an external apparatus using predetermined communication protocols. The predetermined communication protocols may be a known communication protocol such as the Internet protocol and various protocols for implementing the communication protocol, or may be a dedicated protocol defined between the communication circuit 13 and the information processing device 60. The form of the communication line between the communication circuit 13 and the information processing device 60 may be wired, wireless, or a combination of wired and wireless, and may include a public communication line in part.

The controller 12 in the embodiment, for example, acquires the output from the force sensor 20 and the output from the myoelectric sensor 40, synchronizes the output from the force sensor 20 and the output from the myoelectric sensor 40 for each unit time, and transmits the outputs to the information processing device 60 via the communication circuit 13.

In the embodiment, the controller 12 and the communication circuit 13 are provided in the casing 41 (see FIGS. 2 and 6), but the specific manner for implementing the controller 12 and the communication circuit 13 in the detection device 10 is not limited as long as their functions can be achieved.

A battery or the like for supplying and storing power necessary for the operation of the sensor 11, the controller 12, and the communication circuit 13 may be provided in the casing 41. An interface or the like that allows a power line for supplying the power to be coupled to the casing 41 may be further provided in the casing 41.

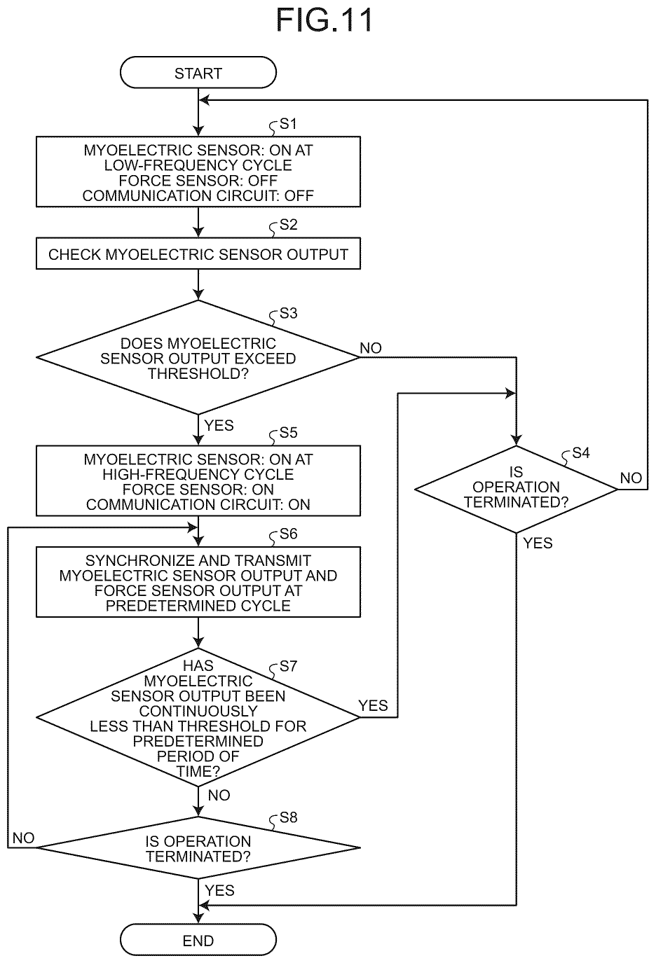

The following describes the process related to the operation of the detection device 10 worn as illustrated in FIG. 6, with reference to FIGS. 11 to 13. The processing described with reference to FIGS. 11 to 13 is implemented by the controller 12 executing the data processing program 12a. In the flowcharts in FIGS. 11 to 13, the operation of the configuration is denoted as “ON” and the non-operation is denoted as “OFF”.

FIG. 11 is a flowchart illustrating an example of a process related to the operation of the detection device 10. After the operation starts, the controller 12 causes the myoelectric sensor 40 to operate at low-frequency cycles (step S1). The controller 12 causes the force sensor 20 and the communication circuit 13 not to operate in the processing at step S1.

The operation at low-frequency cycles in the description with reference to FIGS. 11 to 13 refers to, for example, the operation of performing sensing and producing an output at a time cycle that is 10 times the above unit time. The relation between the low-frequency cycle and the multiplier of the unit time is not limited to this and can be changed as appropriate. However, in the operation at low-frequency cycles, the time interval in sensing is longer than in the operation at high-frequency cycles described below.

The controller 12 checks the output of the myoelectric sensor 40 caused to operate at low-frequency cycles by the processing at step S1 (step S2). The controller 12 determines whether the output of the myoelectric sensor 40 exceeds a threshold by checking the output of the myoelectric sensor 40 in the processing at step S2 (step S3). If it is determined that the output of the myoelectric sensor 40 does not exceed the threshold (No at step S3), the process moves to step S1 unless the operation of the detection device 10 is terminated (No at step S4). In other words, the operating state of the myoelectric sensor 40 at low-frequency cycles and the non-operating state of the force sensor 20 and the communication circuit 13 continue. A condition for the end of operation in the processing at step S4 is, for example, when the user who is the wearer of the detection device 10 performs a terminate operation of the detection device 10 with an application executed on the information processing 60. More specifically, it is assumed that the user performs an operation to start the application before going to sleep and then terminates the application after the user wakes up. The application may be automatically terminated after a preset period of time has elapsed (for example, after 10 hours) since the startup of the application. The condition for ending operation may also be applied to the processing at step S8, the processing at step S14, the processing at step S24, and the processing at step S28 described later.

The threshold in the processing at step S3 and the processing at step S7 described later is, for example, a first threshold Th1 illustrated in FIG. 9. If an output that exceeds the range of the first threshold Th1 is produced, it is determined that the output of the myoelectric sensor 40 exceeds the threshold. If an output that falls within the range of the first threshold Th1 is produced, it is determined that the output of the myoelectric sensor 40 is less than the threshold. An output that overlaps with either one of lines that define the range of the first threshold Th1 may be considered as being neither larger nor less than the threshold, or may be determined to be larger or less than the threshold.

On the other hand, if it is determined that the output of the myoelectric sensor 40 exceeds the predetermined threshold at step S3 (Yes at step S3), the controller 12 causes the myoelectric sensor 40 to operate at high-frequency cycles (step S5). The controller 12 also causes the force sensor 20 and the communication circuit 13 to operate in the processing at step S5. The operation cycles of the force sensor 20 in the processing at step S5 are high-frequency cycles, similar to those of the myoelectric sensor 40.

The operation at high-frequency cycles in the description with reference to FIGS. 11 to 13 refers to, for example, the operation of producing output of sensing at a cycle of the unit time described above. In the embodiment, sensing for one second is performed correspondingly to an output, regardless of whether the sensing is performed at low-frequency cycles or at high-frequency cycles. However, the length of the period in which the sensing is performed in each cycle can be changed as appropriate.

The controller 12 synchronizes the outputs of the myoelectric sensor 40 and the force sensor 20 operating at high-frequency cycles and transmits the outputs to the information processing device 60 at a predetermined cycle (step S6). The predetermined cycle is, for example, a cycle similar to the high-frequency cycle (every second in the embodiment) but not limited to this and can be changed as appropriate. The data generated in this way is transmitted via the communication circuit 13.

The controller 12 determines whether the output of the myoelectric sensor 40 has been continuously less than the threshold for a predetermined period of time (step S7). If it is determined that the output of the myoelectric sensor 40 has not been continuously less than the threshold for a predetermined period of time (No at step S7), the process moves to step S6 unless the operation of the detection device 10 is terminated (No at step S8). In other words, the operating state of the force sensor 20 and the myoelectric sensor 40 at high-frequency cycles, the generation of data, and the transmission of data via the communication circuit 13 continue.

The predetermined period of time in the description with reference to FIGS. 11 to 13 is, for example, five minutes but not limited to this and can be changed as appropriate. However, it is preferred that the predetermined period of time is significantly longer than the unit time of the high-frequency cycle.

On the other hand, if it is determined that the output of the myoelectric sensor 40 has been continuously less than a threshold for the predetermined period of time at step S7 (Yes at step S7), the process moves to step S4. If the operation of the detection device 10 has not been terminated (No at step S4), the process moves to step S1. In other words, the detection device 10 makes a transition to the operating state of the myoelectric sensor 40 at low-frequency cycles and a transition to the non-operating state of the force sensor 20 and the communication circuit 13.

If the operation of the detection device 10 is terminated at step S4 (Yes at step S4) and the operation of the detection device 10 is terminated at step S8 (Yes at step S8), the processing by the controller 12 ends.

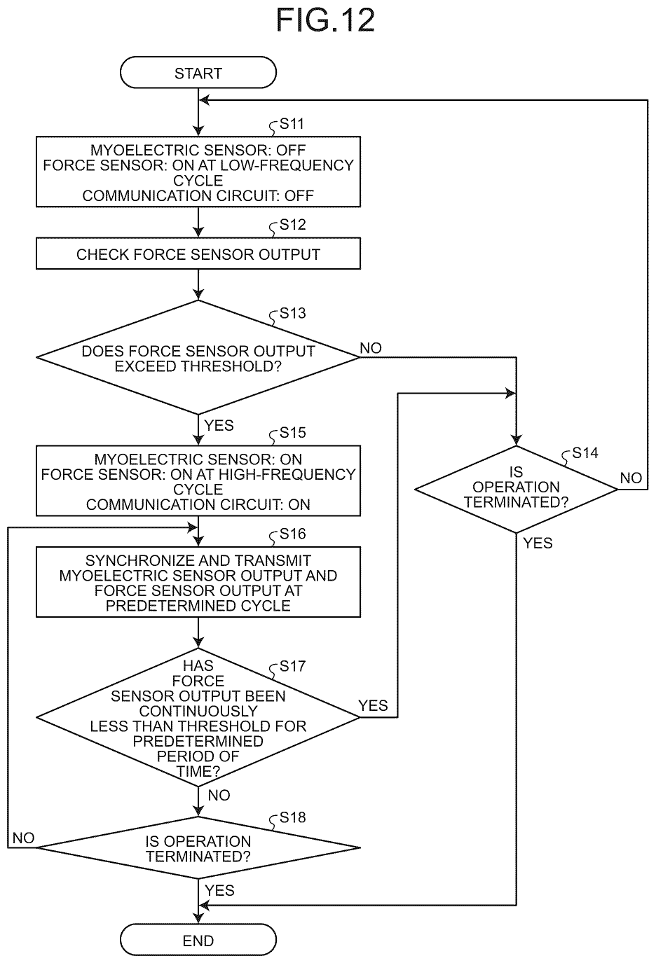

FIG. 12 is a flowchart illustrating an example of a process related to operation of the detection device 10, which is different from that of FIG. 11. After the operation starts, the controller 12 causes the force sensor 20 to operate at low-frequency cycles (step S11). The controller 12 causes the myoelectric sensor 40 and the communication circuit 13 not to operate in the processing at step S11.

The controller 12 checks the output of the force sensor 20 caused to operate at low-frequency cycles by the processing at step S11 (step S12). The controller 12 determines whether the output of the force sensor 20 exceeds a threshold by checking the output of the force sensor 20 in the processing at step S12 (step S13). If it is determined that the output of the force sensor 20 does not exceed the threshold (No at step S13), the process moves to step S11 unless the operation of the detection device 10 is terminated (No at step S14). In other words, the operating state of the force sensor 20 at low-frequency cycles and the non-operating state of the myoelectric sensor 40 and the communication circuit 13 continue.

The threshold in the processing at step S13 and the processing at step S17 described later is, for example, a second threshold Th2 illustrated in FIG. 9. If an output higher than the second threshold Th2 is produced, it is determined that the output of the force sensor 20 exceeds the threshold. If an output that stays below the second threshold Th2 is produced, it is determined that the output of the force sensor 20 is less than the threshold. An output that overlaps with a line that defines the second threshold Th2 may be considered as being neither larger nor less than the threshold, or may be considered as being larger or less than the threshold.

On the other hand, if it is determined that the output of the force sensor 20 exceeds the predetermined threshold at step S13 (Yes at step S13), the controller 12 causes the force sensor 20 to operate at high-frequency cycles (step S15). The controller 12 also causes the myoelectric sensor 40 and the communication circuit 13 to operate in the processing at step S15. The operation cycles of the myoelectric sensor 40 in the processing at step S15 are high-frequency cycles, similar to those of the force sensor 20.

The controller 12 synchronizes the outputs of the myoelectric sensor 40 and the force sensor 20 operating at high-frequency cycles and transmits the outputs to the information processing device 60 at a predetermined cycle (step S16).

The controller 12 determines whether the output of the force sensor 20 has been continuously less than the threshold for a predetermined time (step S17). If it is determined that the output of the force sensor 20 has not been continuously less than the threshold for a predetermined period of time (No at step S17), the process moves to step S16 unless the operation of the detection device 10 is terminated (No at step S18). In other words, the operating state of the force sensor 20 and the myoelectric sensor 40 at high-frequency cycles, the generation of data, and the transmission of data via the communication circuit 13 continue.

On the other hand, if it is determined that the output of the force sensor 20 has been continuously less than a threshold for the predetermined period of time at step S17 (Yes at step S17), the process moves to step S14. If the operation of the detection device 10 has not been terminated (No at step S14), the process moves to step S11. In other words, the detection device 10 makes a transition to the operating state of the force sensor 20 at low-frequency cycles and a transition to the non-operating state of the myoelectric sensor 40 and the communication circuit 13.

If the operation of the detection device 10 is terminated at step S14 (Yes at step S14) and the operation of the detection device 10 is terminated at step S18 (Yes at step S18), the processing by the controller 12 ends.

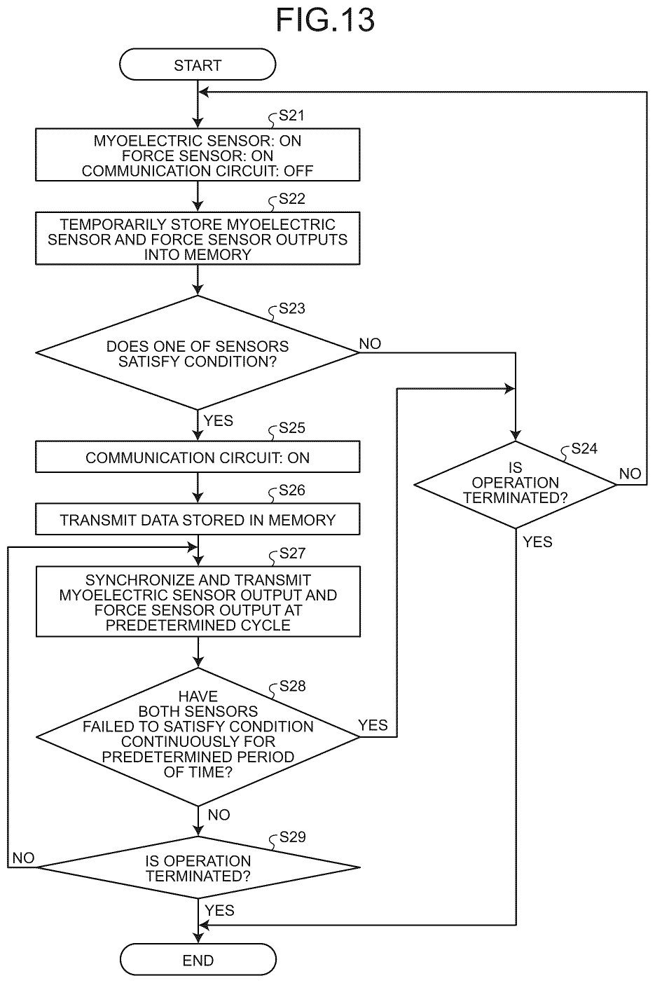

FIG. 13 is a flowchart illustrating an example of a process related to operation of the detection device 10, which is different from those of FIG. 11 and FIG. 12. After the operation starts, the controller 12 causes the force sensor 20 and the myoelectric sensor 40 to operate (step S21). The controller 12 causes the communication circuit 13 not to operate in the processing at step S21.

The operation cycle of each of the force sensor 20 and the myoelectric sensor 40 in the processing at step S21 may be a low-frequency cycle or a high-frequency cycle.

The controller 12 generates data in which the outputs of the force sensor 20 and the myoelectric sensor 40 caused to operate by the processing at step S21 are synchronized, and temporarily stores the data in the memory 12b (step S22). In the operation of temporarily storing the data in the memory 12b in the processing at step S22, for example, only the latest five minutes of data is retained and data after a lapse of five minutes is discarded. The length of the retention period can be changed as appropriate.

The controller 12 determines whether one of the force sensor 20 and the myoelectric sensor 40 satisfies a condition (step S23). The conditions referred to in the processing at step S23 and the processing at step S28 described later are as follows: in the case of the force sensor 20, for example, when the output of the force sensor 20 exceeds a predetermined threshold, the condition is determined to being satisfied, in the same manner as in the determination at step S13 described above; in the case of the myoelectric sensor 40, when the output of the myoelectric sensor 40 exceeds a predetermined threshold, the condition is determined to being satisfied, in the same manner as in the determination at step S3 described above.

If it is determined that neither of the sensors satisfies the condition at step S23 (No at step S23), the process moves to step S21 unless the operation of the detection device 10 is terminated (No at step S24). In other words, the operation of the force sensor 20 and the myoelectric sensor 40 and the operation of temporarily storing data in the memory 12b continue.

On the other hand, if it is determined that one of the sensors satisfies the condition at step S23 (Yes at step S23), the controller 12 causes the communication circuit 13 to operate (step S25). The controller 12 transmits the data temporarily stored in the memory 12b in the processing at step S22 to the information processing device 60 via the communication circuit 13 (step S26).

If the force sensor 20 and the myoelectric sensor 40 are operating at low-frequency cycles at step S21, the operation cycles of the force sensor 20 and the myoelectric sensor 40 become high-frequency cycles at the point in time at step S25.

The controller 12 synchronizes the outputs of the myoelectric sensor 40 and the force sensor 20 and transmits the outputs to the information processing device 60 at a predetermined cycle (step S27).

The controller 12 determines whether neither the force sensor 20 nor the myoelectric sensor 40 has satisfied the condition continuously for a predetermined period of time (step S28). If it is not determined that both sensors have failed to satisfy the condition continuously for a predetermined period of time (No at step S28), the process moves to step S27 unless the operation of the detection device 10 is terminated (No at step S29). In other words, the operating state of the force sensor 20 and the myoelectric sensor 40 at high-frequency cycles, the generation of data, and the transmission of data via the communication circuit 13 continue.

On the other hand, if it is determined that neither the force sensor 20 nor the myoelectric sensor 40 has satisfied the condition continuously for a predetermined period of time at step S28 (Yes at step S28), the process moves to step S24. If the operation of the detection device 10 has not been terminated (No at step S24), the process moves to step S21. In other words, the detection device 10 transitions to a state in which the operation of the force sensor 20 and the myoelectric sensor 40 and the operation of temporarily storing data in the memory 12b are performed.

If the operation of the detection device 10 is terminated at step S24 (Yes at step S24) and the operation of the detection device 10 is terminated at step S29 (Yes at step S29), the processing by the controller 12 ends.

Three processes performed by the controller 12 have been exemplarily described with reference to FIGS. 11 to 13. The controller 12 in the embodiment operates according to any of these three processes. The process to be applied may be determined in advance or may be selectable. For example, the detection device 10 may be provided such that the process to be applied to the controller 12 can be changed through an operation on an operation device such as a switch 46 illustrated in FIG. 6. The process to be applied to the controller 12 may be changeable through an operation on an operation device 63 of the information processing device 60 and communication between a communication circuit 62 and the communication circuit 13.

The following describes the configuration of the information processing device 60 and the processing performed by the information processing device 60 with reference to FIG. 1 and FIGS. 14 to 21.

The information processing device 60 includes a data acquisition processor 61, an operation device 63, a display 64, an arithmetic unit (arithmetic circuit) 70, and a storage 80. The data acquisition processor 61 acquires data output from the sensor 11 from the detection device 10. The data acquisition processor 61 illustrated in FIG. 1 includes a communication circuit 62. The communication circuit 62 performs processing related to communication with an external apparatus such as the detection device 10. The communication circuit 62 has a circuit or the like to function as an NIC, in the same manner as the communication circuit 13. In the embodiment, a communication protocol employed by the information processing device 60 and a communication protocol employed by the communication circuit 13 are selected so that communication is established between the information processing device 60 and the communication circuit 13. The data acquisition processor 61 acquires data transmitted from the detection device 10 via the communication circuit 13, through communication between the communication circuit 13 and the communication circuit 62. In FIG. 1, training data D1 and distinction target data D4 are illustrated as the data acquired by the data acquisition processor 61.

The operation device 63 receives inputs to the information processing device 60 from a user. Specifically, the operation device 63 has a human interface, such as a keyboard and a mouse, and receives the content of input operation performed by the user via the human interface. The operation device 63 may be a touch panel or the like integrated with the display 64.

The display 64 performs display output in accordance with the content of processing performed in the information processing device 60. Specifically, the display 64 includes, for example, at least one or more display devices, such as a liquid crystal display and an organic electroluminescence (EL) display, and performs display output by such display devices. The display output by the display 64 depends on the content of processing by the arithmetic unit 70.

The arithmetic unit 70 performs various information processing performed in the information processing device 60. Specifically, the arithmetic unit 70 has an arithmetic circuit such as a central processing unit (CPU). The arithmetic unit 70 in the embodiment functions as a feature acquisition processor 71, a learning processor 73, and a distinction processor 75. In FIG. 1, a filter 72, a model learning program 74, and a bruxism distinction program 76 are illustrated in the arithmetic unit 70. The filter 72 is used when the arithmetic unit 70 functions as the feature acquisition processor 71, the model learning program 74 is used when the arithmetic unit 70 functions as the learning processor 73, and the bruxism distinction program 76 is used when the arithmetic unit 70 functions as the distinction processor 75. The filter 72, the model learning program 74, and the bruxism distinction program 76 may be implemented as hardware in the arithmetic unit 70, or may be stored as a software program that can be read and referred to in the arithmetic unit 70. The software program may be stored in the storage 80 and read from the storage 80 by the arithmetic unit 70 in the processing corresponding to each function.

The storage 80 stores a software program and data to be used in information processing by the arithmetic unit 70. Specifically, the storage 80 includes, for example, at least one or more storage devices, such as a hard disk drive (HDD) and a solid state drive (SSD). FIG. 1 illustrates training data D1, feature data D2, model data D3, and distinction target data D4 as the data stored in the storage 80.

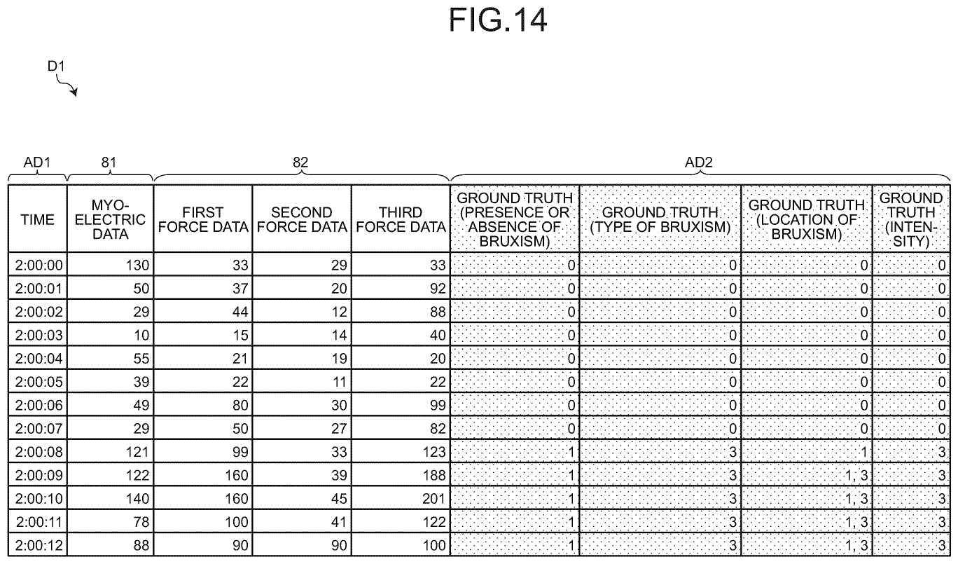

The following describes the information processing by the arithmetic unit 70 and the data stored in the storage 80 with reference to FIGS. 14 to 21.

FIG. 14 is a schematic diagram illustrating an example of the training data D1. As illustrated in FIG. 14, the training data D1 includes myoelectric data 81, force data 82, synchronization information AD1, and distinction information AD2. The myoelectric data 81 is a column in which a parameter corresponding to the output of the myoelectric sensor 40 is registered. In the force data 82, a parameter corresponding to the output of the force sensor 20 is registered. The force data 82 illustrated in FIG. 14 includes three columns: “first force data”, “second force data”, and “third force data”. The three columns correspond to outputs of sensors different from each other. For example, the “first force data” corresponds to the output of the first sensor element 221, the “second force data” corresponds to the output of the second sensor element 222, and the “third force data” corresponds to the output of the third sensor element 223. In other words, the number of columns included in the force data 82 corresponds to the number of sensors provided in the force sensor 20. The outputs of the sensor 11 described above are transmitted from the detection device 10 via the communication circuit 13, whereby the myoelectric data 81 and the force data 82 are acquired by the information processing device 60 via the communication circuit 62 of the data acquisition processor 61.

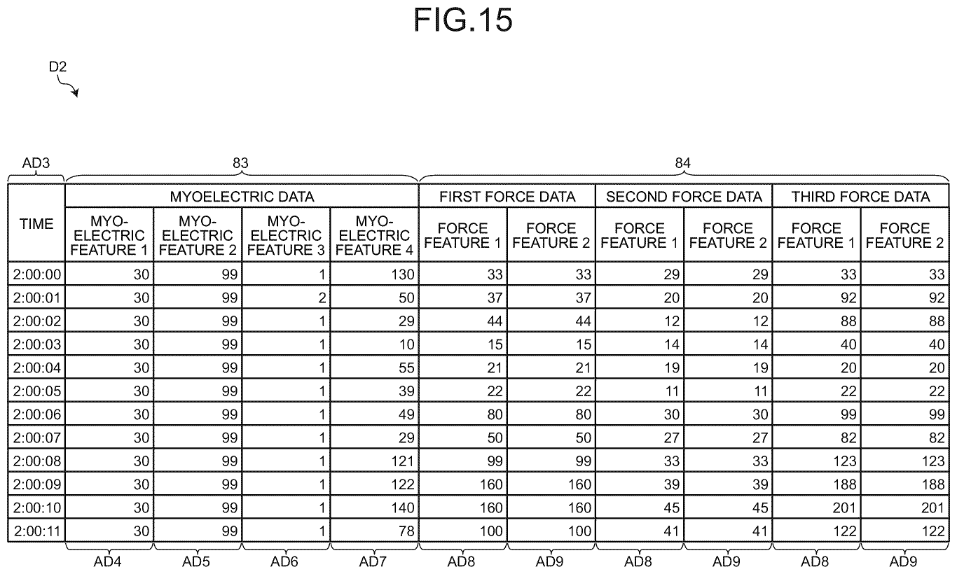

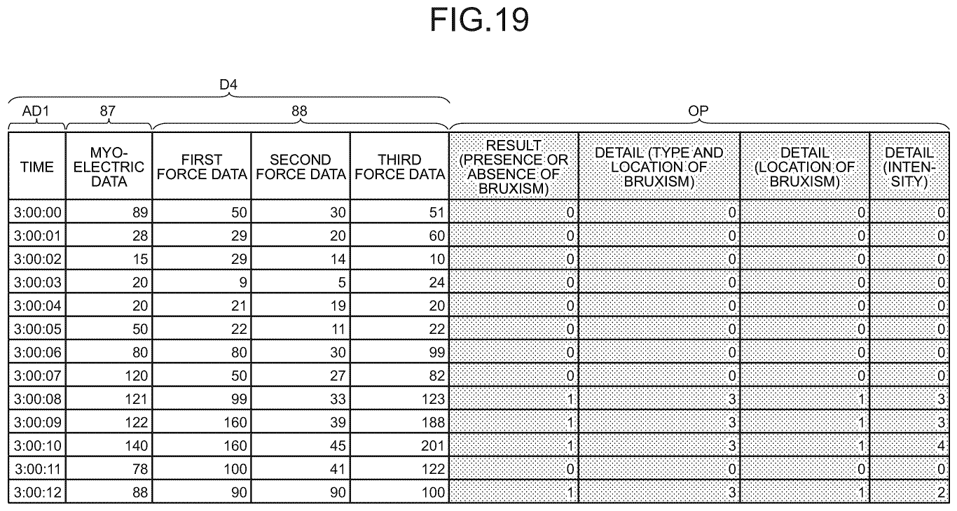

The synchronization information AD1 is a column in which a parameter of time information indicating the point in time of the acquisition of the output of the myoelectric sensor 40 registered in the myoelectric data 81 and the output of the force sensor 20 registered in the force data 82 is registered. A plurality of records in the table illustrated in FIG. 14 individually have different parameters of time information registered in the fields of the synchronization information AD1. In other words, the training data D1 illustrated in FIG. 14 is data in a format allowing the storage to keep information for each of the points in time when the outputs of the force sensor 20 and the myoelectric sensor 40 are acquired, on a record basis. The format of data illustrated in FIGS. 15 and 19 described later is similar to the format of the training data D1. In other words, since myoelectric data (for example, myoelectric data 81) and force data (for example, force data 82) can be associated with each other using time information corresponding to data acquisition timing, such as the synchronization information AD1, it can be said that the myoelectric data and the force data are data in which the output of the force sensor 20 is synchronized with the output of the myoelectric sensor 40. The output of the force sensor 20 and the output of the myoelectric sensor 40 are synchronized when data transmission via the communication circuit 13 is performed under the control of the controller 12. The timing of adding the synchronization information AD1 may be timing before the transmission of data via the communication circuit 13 or after the transmission of data via the communication circuit 13.

The processing of adding the synchronization information AD1 to data may be performed by the controller 12, but in the embodiment, the processing is performed by the data acquisition processor 61 of the information processing device 60.

The distinction information AD2 is a column in which a parameter indicating the relation between bruxism and a combination of the output of the force sensor 20 and the output of the myoelectric sensor 40 indicated by each record in the training data D1 is registered. Specifically, the distinction information AD2 illustrated in FIG. 14 includes four columns: “ground truth (presence or absence of bruxism)”, “ground truth (type of bruxism)”, “ground truth (location of bruxism)”, and “ground truth (intensity)”.

The parameter registered in a field included in the column “ground truth (presence or absence of bruxism)” indicates whether bruxism is occurring at the point in time corresponding to the record containing the parameter. Hereafter, the expression “parameter of A” means a parameter registered in a field included in the column A. For example, if a combination of the parameter of myoelectric data 81 and the parameters of force data 82 in a certain record is a combination of parameters generated “when bruxism is occurring”, such as in periods T1 and T2 in FIG. 9, the parameter of “ground truth (presence or absence of bruxism)” in this record is “1”. Conversely, if a combination of the parameter of myoelectric data 81 and the parameters of force data 82 is a combination of parameters generated “when bruxism is not occurring”, the parameter of “ground truth (presence or absence of bruxism)” in this record is “0”.

The parameter of “ground truth (type of bruxism)” indicates the type of bruxism. For a record in which the parameter of “ground truth (presence or absence of bruxism)” is “0”, “0” is registered as the parameter of “ground truth (type of bruxism)”. In other words, in a record corresponding to the point in time when bruxism is not occurring, “0” is registered to indicate there is no bruxism type to be presented because no bruxism is occurring in the first place.

Among the parameters of “ground truth (type of bruxism)”, the number of variations of parameters that are not “0” corresponds to the types of bruxism that can be distinguished by the detection system 100. For example, if there are four types of bruxism that can be distinguished by the detection system 100, four parameters, such as “1”, “2”, “3”, and “4”, are assumed as parameters corresponding to the types of bruxism. In this case, therefore, any of “0”, “1”, “2”, “3”, or “4” is set as the parameter of “ground truth (type of bruxism)”.

For example, the parameter “1” of “ground truth (type of bruxism)” indicates that the type of bruxism is grinding. The parameter “2” of “ground truth (type of bruxism)” indicates that the type of bruxism is tapping. The parameter “3” of “ground truth (type of bruxism)” indicates that the type of bruxism is clenching. The parameter “4” of “ground truth (type of bruxism)” indicates that the type of bruxism is gnashing. Such a relation between numbers and the types of bruxism is only an example and is not limited to this, and other numbers and other types may be further added. The types of bruxism may reflect medical information recognized at present and in the future.

Grinding is bruxism that occurs when upper and lower teeth are ground from side to side. Grinding tends to occur more frequently during human sleep and may be accompanied by sound when it occurs. The onomatopoeia expression for such sound is, for example, “gritting”. It is believed that when grinding, humans tend to unconsciously move their teeth faster and in larger movements.

Tapping is bruxism that occurs when the upper and lower teeth make contact. When tapping, humans tend to move the lower jaw up and down. The onomatopoeia expression for the sound produced by tapping is, for example, “clicking”.

Clenching is bruxism that occurs when the upper and lower teeth are held together tightly. Humans who habitually grit their teeth at work, in sports, or in other situations tend to relatively often produce clenching. When clenching occurs, the jaw is under a great strain. Humans with a tendency to produce clenching may unconsciously hold their teeth together during sleep and experience stiffness when yawning upon awakening. These humans may show a tendency to feel fatigue upon awakening.

Gnashing is bruxism that occurs when specific portions of the teeth are ground together. Humans with a tendency to produce gnashing may gnash their teeth during sleep.

The parameter of “ground truth (location of bruxism)” indicates the location where bruxism is occurring. For a record in which the parameter of “ground truth (presence or absence of bruxism)” is “0”, “0” is registered as the parameter of “ground truth (location of bruxism)”.

Specifically, the parameters of “ground truth (location of bruxism)” that are not “0” indicate the sensor by which the occurrence of bruxism is distinguished, among the plurality of sensors that function as the force sensor 20. More specifically, among the parameters of “ground truth (type of bruxism)”, the number of variations of parameters that are not “0” corresponds to the number of sensors that function as the force sensor 20. For example, if three sensors are provided in total which function as the force sensor 20, namely, the first sensor element 221, the second sensor element 222, and the third sensor element 223, it is assumed that one or more of the three parameters, such as “1”, “2”, and “3”, are registered in a field of “ground truth (location of bruxism)” as the parameter indicating the location where bruxism is occurring. The parameter of “ground truth (location of bruxism)” in a record corresponding to the point in time when bruxism is occurring at multiple locations, includes multiple values.

For example, the parameter “1” of “ground truth (location of bruxism)” indicates the location where the first sensor element 221 is provided, that is, indicates that bruxism is occurring near the molar M1 in FIG. 7. The parameter “2” of “ground truth (location of bruxism)” indicates the location where the second sensor element 222 is provided, that is, indicates that bruxism is occurring near the molar M2 in FIG. 7. The parameter “3” of “ground truth (location of bruxism)” indicates the location where the third sensor element 223 is provided, that is, indicates that bruxism is occurring near the incisors M3 in FIG. 7. Based on these examples, if the parameter of “ground truth (location of bruxism)” is “1, 3”, it means that bruxism is occurring near the molar M1 and near the incisors M3 in FIG. 7.

The parameter of “ground truth (intensity)” indicates the degree of strength of bruxism. For a record in which the parameter of “ground truth (presence or absence of bruxism)” is “0”, “0” is registered as the parameter of “ground truth (intensity)”. Specifically, in the case where the numerical value of the parameter of “ground truth (intensity)” is not “0”, a higher numerical value indicates a higher strength of bruxism. The numerical range of parameters of “ground truth (intensity)” can be set as desired.

The data acquisition processor 61 acquires, from the detection device 10, data output from the sensor 11, and whereby the myoelectric data 81 and the force data 82 in the training data D1 are registered. The synchronization information AD1 in the training data D1 is automatically added through the processing by a circuit included in the data acquisition processor 61 or the arithmetic unit 70 when data is acquired by the data acquisition processor 61. The distinction information AD2 in the training data D1 is manually added by a human who is able to diagnose bruxism, for example, a dentist or a person instructed by a dentist. In adding the distinction information AD2, for example, the human individually determines when bruxism starts and when the bruxism ends, while the display 64 is performing display output indicating the myoelectric data 81 and the force data 82 (see, for example, FIG. 9). The human makes an input in accordance with the determination result via the operation device 63. The distinction information AD2 is thus added.

With the addition of the distinction information AD2 to the myoelectric data 81 and the force data 82, the training data D1 functions as training data in machine learning.

The feature acquisition processor 71 generates the feature data D2 from the training data D1. The following describes the configuration of the feature data D2 and the processing performed by the feature acquisition processor 71 with reference to FIG. 15.

FIG. 15 is a schematic diagram illustrating an example of the feature data D2. The content of the feature data D2 illustrated in FIG. 15 corresponds to the content of the training data D1 illustrated in FIG. 14. As illustrated in FIG. 15, the feature data D2 includes myoelectric feature data 83, force feature data 84, and synchronization information AD3. In the myoelectric feature data 83, a parameter of a feature corresponding to the output of the myoelectric sensor 40 is registered. The myoelectric feature data 83 includes first myoelectric feature AD4, second myoelectric feature AD5, third myoelectric feature AD6, and fourth myoelectric feature AD7. The first myoelectric feature AD4, the second myoelectric feature AD5, the third myoelectric feature AD6, and the fourth myoelectric feature AD7 represent features derived from the myoelectric data 81 through different processes.

The parameter of the first myoelectric feature AD4 is the root mean square (RMS) of a myoelectric parameter after preprocessing. The preprocessing will be described later. Specifically, the parameter of the first myoelectric feature AD4 is derived based on the following equation (1). Here, m (t) denotes myoelectric data after preprocessing at time t as a certain timing. i (t) denotes the number of a myoelectric data vector (one-dimensional array) at time t. In equations (1) to (4), n corresponds to the output frequency of the myoelectric sensor 40. In the embodiment, n=512 is satisfied, since the myoelectric sensor 40 produces an output 512 times per second (512 Hz) as described above. Therefore, m (i) on the left side of equation (1) can be a value within a range from m(1) to m(512). Each of values from m(1) to m(512) is a value corresponding to one of 512 outputs (512 Hz) of the myoelectric sensor 40 obtained in one second.

$${m}_{1} ( t ) = \sqrt{\frac{1}{n} {\sum }_{i = 1}^{n} {m ( i )}^{2}} \qquad ( 1 )$$

The parameters of the myoelectric data 81 illustrated in FIG. 14 are only outlines, and in practice, parameters indicating data corresponding to the output frequency of the myoelectric sensor 40 are registered as described above. The parameter of the second myoelectric feature AD5 indicates an average rectified value (ARV) of the myoelectric parameter after preprocessing. Specifically, a low-pass filter H₂ (ω) is applied to the absolute value of the myoelectric parameter, and a value after passing through the low-pass filter H₂ (ω) is registered as the parameter of the second myoelectric feature AD5 as expressed by the following equation (2). The cutoff frequency of the low-pass filter H₂ (ω) is, for example, 2 Hz, but can be changed as appropriate. Here, the value after passing through the low-pass filter H₂ (ω) corresponds to data obtained by filtering the output of the myoelectric sensor 40 to a specific frequency component.

$${m}_{2} ( t ) = {\sum }_{k} {h}_{2} ( k ) \vert m ( i ( t ) - k ) \vert \qquad ( 2 )$$

The parameter of the third myoelectric feature AD6 indicates a value after passing through a bandpass filter H₃ (ω) applied to the myoelectric parameter after preprocessing. The value after passing through the bandpass filter H₃ (ω) is expressed by the following equation (3). The passband by the bandpass filter H₃ (ω) is, for example, 50 Hz to 200 Hz, but can be changed as appropriate. Here, the value after passing through the bandpass filter H₃ (ω) corresponds to data obtained by filtering the output of the myoelectric sensor 40 to a specific frequency component.

$${m}_{3} ( t ) = {\sum }_{k} {h}_{3} ( k ) m ( {n}_{m} ( t ) - k ) \qquad ( 3 )$$

The parameter of the fourth myoelectric feature AD7 indicates a value after passing through a bandpass filter H₄ (ω) applied to the myoelectric parameter after preprocessing. The bandpass filter H₃ (ω) and the bandpass filter H₄ (ω) have different passbands. The value after passing through the bandpass filter H₄ (ω) is expressed by the following equation (4). The passband of the bandpass filter H₄ (ω) is, for example, 200 Hz to 300 Hz, but can be changed as appropriate. Here, the value after passing through the bandpass filter H₄ (ω) corresponds to data obtained by filtering the output of the myoelectric sensor 40 to a specific frequency component.

$${m}_{4} ( t ) = {\sum }_{k} {h}_{4} ( k ) m ( {n}_{m} ( t ) - k ) \qquad ( 4 )$$

When the parameter of the third myoelectric feature AD6 is represented in a frequency domain, M₃ (ω)=H₃ (ω) M (ω) is satisfied. M (ω) and M₃ (ω) are obtained by Fourier transform of m (t) and m₃(t), respectively. When the parameter of the fourth myoelectric feature AD7 is represented in a frequency domain, M₄ (ω)=H₄ (ω) M (ω) is satisfied. M (ω) in these expressions represents the frequency domain of the myoelectric data 81 after preprocessing.

The force feature data 84 includes a first force feature AD8 and a second force feature AD9. The first force feature AD8 and the second force feature AD9 represent features derived from the force data 82 through different processes.

The parameter of the first force feature AD8 indicates a feature normalized by the difference between the value of the parameter of the force data 82 and the value in a steady state p₀. The value of the parameter of the first force feature AD8 is expressed by the following equation (5). Here, p (t) denotes the value of the parameter of the force data 82 at time t as a certain timing. The value in a steady state p₀ is the value corresponding to the output of the force sensor 20 in a static state where no force is applied to the first section P1.

$${p}_{1} ( t ) = \frac{p ( t ) - {p}_{0}}{{p}_{0}} \qquad ( 5 )$$

The parameter of the second force feature AD9 indicates a value after passing through a moving average filter H₆ (ω) applied to the value (p₁) of the parameter of the first force feature AD8. It can be said that the value of the parameter of the second force feature AD9 is a value time-averaged over a unit time (for example, 1 second) that delimits a plurality of records of the training data D1. The value after passing through the moving average filter H₆ (ω) is expressed by the following equation (6). Here, nₚ (t) denotes the number of a force data vector (one-dimensional array) at time t. In equation (6), n corresponds to the output frequency of the force sensor 20. In the embodiment, n=8 is satisfied, since the force sensor 20 produces an output 8 times per second (8 Hz) as described above. Here, the value after passing through the moving average filter H₆ (ω) corresponds to data obtained by filtering the output of the force sensor 20 to a specific frequency component.

$${p}_{2} ( t ) = {\sum }_{k} {h}_{6} ( k ) {p}_{1} ( {n}_{p} ( t ) - k ) \qquad ( 6 )$$

When the parameter of the second force feature AD9 is represented in a frequency domain, P₂ (ω)=H₆ (ω) P₁ (ω) is satisfied. P₁ (ω) represents the frequency domain of the first force feature AD8 and corresponds to the Fourier transform of p₁ (t).

Although the myoelectric data 81 and the force data 82 illustrated in FIG. 14 and other drawings are described in outline as numerical values, the actual myoelectric data 81 and force data 82 can be considered as waveforms representing increase or decrease of outputs, as described with reference to FIG. 9. The components contained in the waveforms therefore can be expressed in frequency domains as described above, such as equations (2), (3), (4), and (6), M₃ (ω)=H₃ (ω) M (ω), M₄ (ω)=H₄ (ω) M (ω), and P₂ (ω)=H₆ (ω) P₁ (ω); and the components included in the specific frequency domains that can be expressed in such a way can be targets to be acquired by the feature acquisition processor 71.

The first force feature AD8 and the second force feature AD9 are derived individually for each of a plurality of columns in the force data 82 of the training data D1 (see FIG. 14). In other words, the first force feature AD8 and the second force feature AD9 are derived individually for each of the sensors that function as the force sensor 20. For example, the parameters of the first force feature AD8 and the second force feature AD9 for “force sensor 1” illustrated in FIG. 15 are derived from the parameter registered in the column “force sensor 1” of the force data 82 illustrated in FIG. 14. The parameters of the first force feature AD8 and the second force feature AD9 for “force sensor 2” illustrated in FIG. 15 are derived from the parameter registered in the column “force sensor 2” of the force data 82 illustrated in FIG. 14. The parameters of the first force feature AD8 and the second force feature AD9 for “force sensor 3” illustrated in FIG. 15 are derived from the parameter registered in the column “force sensor 3” of the force data 82 illustrated in FIG. 14.

The synchronization information AD3 in the feature data D2 (see FIG. 15) is a parameter corresponding to the synchronization information AD1 in the training data D1 (see FIG. 14). In other words, a record of the feature data D2 illustrated in FIG. 15 indicates the features of the myoelectric data 81 and the force data 82 included in a record of the training data D1 that includes the synchronization information AD1 having the same parameter as the parameter of the synchronization information AD3 in the record of feature data D2.

The feature acquisition processor 71 performs preprocessing on the parameter of the myoelectric data 81 prior to deriving the first myoelectric feature AD4, the second myoelectric feature AD5, the third myoelectric feature AD6, and the fourth myoelectric feature AD7. The above “myoelectric parameter after preprocessing” refers to the parameter of the myoelectric data 81 after the preprocessing is performed. Specifically, the parameter of the myoelectric data 81 can be regarded as a parameter of an electromyographic signal. The effective frequency bandwidth of the electromyographic signal is from 5 Hz to 500 Hz. The feature acquisition processor 71 applies a bandpass filter with the effective frequency bandwidth to the parameter of the myoelectric data 81, and regards the parameter after passing through the bandpass filter as “myoelectric parameter after preprocessing”.

Furthermore, since each subject that is a human undergoing the sensing by the detection device 10 has a different muscle tissue, a myoelectric potential signal measured also differs from subject to subject even when they make the same movement. Even for the same subject, measured values may vary depending on their physical condition such as a skin condition. Therefore, skin resistance may vary due to experiments across days, attachment and removal of the electrodes of the myoelectric sensor 40, and the like, whereby the values measured may also vary. From this perspective, it is preferable to use normalized myoelectric potential values. For example, a maximum voluntary contraction (% MVC) method can be employed in normalization. In this case, as a preliminary preparation for the sensing by the detection device 10, the subject may undergo MVC to obtain an integrated electromyogram (IEMG), and the processing of normalizing the parameter of the myoelectric data 81 using the value of the IEMG as a value of 100% may be further performed as a processing included in the preprocessing.

The feature acquisition processor 71 illustrated in FIG. 1 derives the first myoelectric feature AD4, the second myoelectric feature AD5, the third myoelectric feature AD6, and the fourth myoelectric feature AD7 of the myoelectric feature data 83, based on “the myoelectric parameter after preprocessing” obtained through the preprocessing described above. The feature acquisition processor 71 also derives the first force feature AD8 and the second force feature AD9 of the force feature data 84, based on the parameter of the force data 82. The feature acquisition processor 71 generates records of the feature data D2 in units of records of the training data D1 with the synchronization information AD1, and generates the feature data D2 by adding the synchronization information AD3 to each record of the feature data D2.

The filter 72 is, for example, but not limited to, a component obtained by executing a software program that functions as various filters used in the preprocessing and in the derivation of the second myoelectric feature AD5, the third myoelectric feature AD6, the fourth myoelectric feature AD7, and the second force feature AD9. For example, the feature acquisition processor 71 may be a dedicated circuit and the filter 72 may be provided as a digital filter. In the embodiment, the filter 72 is a component obtained by executing a software program that further includes the content of processing for deriving the first myoelectric feature AD4 and the first force feature AD8. However, among the processings by the feature acquisition processor 71, a software program using no filter and the filter 72 may be provided separately.

The learning processor 73 performs machine learning to derive the model data D3, based on the distinction information AD2 of the training data D1 (see FIG. 14) and the feature data D2 (see FIG. 15). Specifically, for example, the learning processor 73 functions when the arithmetic unit 70 executes the model learning program 74. The learning processor 73 performs supervised machine learning based on support vector machines (SVM). SVM is a pattern recognition model for classifying patterns based on combinations of multiple parameters. Applicable machine learning methods are not limited to SVM. Any of supervised machine learning methods such as decision trees and logistic regression can be applied.

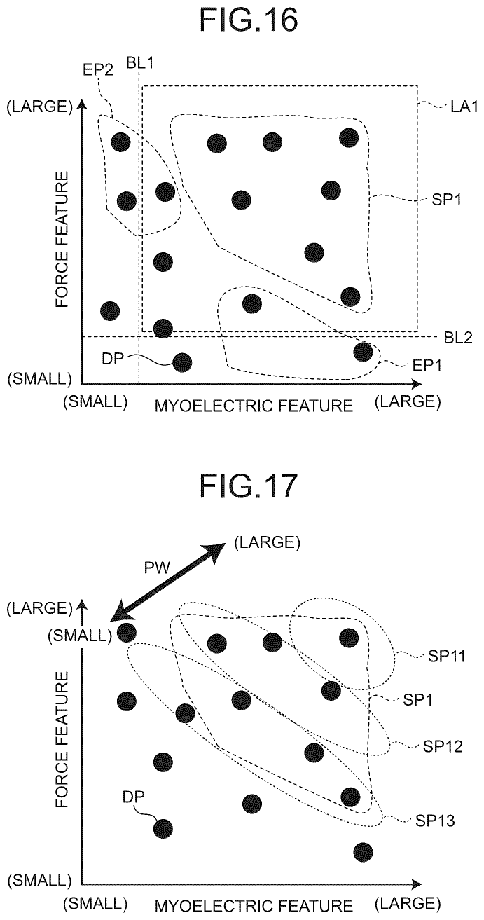

In the embodiment, the distinction information AD2 is used as the basis of classification to implement supervised machine learning. In other words, the distinction information AD2 indicates which combination of parameters corresponds to which pattern, in terms of “presence or absence of bruxism”, “type of bruxism”, “location of bruxism”, and “intensity of bruxism”. Since the training data D1 and the feature data D2 can be associated with each other using the synchronization information AD1 and the synchronization information AD3, the relation of the myoelectric feature data 83 and the force feature data 84 to the distinction information AD2 can be identified. In the supervised machine learning using SVM in the embodiment, therefore, a model is generated to classify combinations of the parameters of the myoelectric feature data 83 and the parameters of the force feature data 84 into the plurality of patterns, in terms of “presence or absence of bruxism”, “type of bruxism”, “location of bruxism”, and “intensity of bruxism”. The model is the model data D3 illustrated in FIG. 1. The following describes an overview of the machine learning with reference to FIGS. 16 to 18.

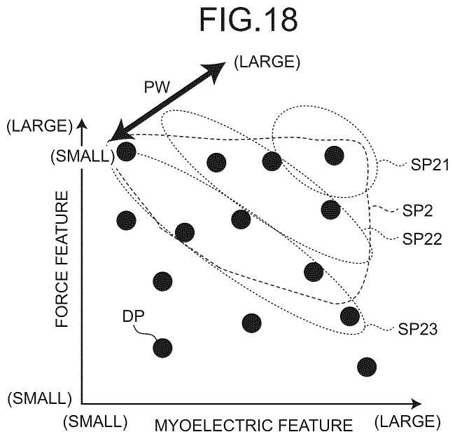

FIG. 16 is a two-dimensional graph schematically illustrating machine learning related to classification of the presence or absence of bruxism. The vertical axis in FIG. 16, and FIGS. 17 and 18 described later represents a force feature. As used herein, the force feature is either the first force feature AD8 or the second force feature AD9. The horizontal axis in FIGS. 16, 17, and 18 represents a myoelectric feature. As used herein, the myoelectric feature is any one of the first myoelectric feature AD4, the second myoelectric feature AD5, the third myoelectric feature AD6, or the fourth myoelectric feature AD7. The points DP in FIGS. 16, 17, and 18 each represent a combination of force features and myoelectric features of a certain record in the feature data D2. In FIGS. 16, 17, and 18, one of a plurality of black dots is denoted by a sign DP, but the other black dots also each represent a combination of force features and myoelectric features of a certain record in the feature data D2. The term “point DP” therefore indicates a combination of force features and myoelectric features of a certain record in the feature data D2.

In FIGS. 16, 17 and 18, the relation between one force feature and one myoelectric feature is illustrated for the sake of clarity of the drawings, but in practice, the processing is performed assuming a {(q×α)+β}-dimensional vector space based on a combination of the first force feature AD8 and the second force feature AD9 of each of the plurality of sensors, and the first myoelectric feature AD4, the second myoelectric feature AD5, the third myoelectric feature AD6, and the fourth myoelectric feature AD7. As used herein, “q” is the number of parameter types included in the force feature data 84. “α” is the number of sensors that function as the force sensor 20. “β” is the number of parameter types included in the myoelectric feature data 83. In the embodiment, therefore, q=2, α=3, and β=4 are satisfied. Based on this, in the embodiment, {(q×α)+β}={(2×3)+4}=10-dimensional vector space is assumed.

FIG. 16 illustrates the classification of “presence or absence of bruxism” as an example of classifications by SVM, among the classifications included in the distinction information AD2. The classification of “presence or absence of bruxism” is the classification based on the value of “ground truth (presence or absence of bruxism)”. In FIGS. 16 and 17, a point DP within a boundary SP1 is a point DP with a value of 1 for “ground truth (presence or absence of bruxism)”, that is, a point DP corresponding to a combination of force features and myoelectric features in a case where bruxism is occurring. The boundary SP1 is depicted as a two-dimensional frame line in FIG. 16, but in practice a hyperplane in the {(q×α)+β}-dimensional vector space.

Although not included in the distinction information AD2, information indicating other classifications may be further included in the training data, which allows other classifications. For example, the area within a boundary EP1 illustrated in FIG. 16 corresponds to the first pattern described with reference to FIG. 10. The area within a boundary EP2 illustrated in FIG. 16 corresponds to the second pattern described with reference to FIG. 10. When the distinction information AD2 further includes parameters indicating whether these patterns are applicable, it is also possible to perform machine learning for the classifications such as the boundary EP1 and the boundary EP2.

It is also possible to exclude some of the data included in the feature data D2 that clearly do not correspond to bruxism. As described with reference to FIG. 9, bruxism produces characteristic outputs in both the force sensor 20 and the myoelectric sensor 40. On the other hand, a point DP where the myoelectric feature is smaller than that of a boundary line BL1 illustrated in FIG. 16 clearly has a poor output of the myoelectric sensor 40. A point DP where the force feature is smaller than that of a boundary line BL2 illustrated in FIG. 16 clearly has a poor output of the force sensor 20. From this perspective, at least one of the point DP where the myoelectric feature is smaller than that of the boundary line BL1 and the point DP where the force feature is smaller than that of the boundary line BL2 may be defined as an exclusion target that is excluded from the targets of machine learning. In this case, the processing load related to machine learning can be further reduced. Even when using data obtained by applying variable operational control of the detection device 10 based on thresholds (for example, the first threshold Th1, the second threshold Th2) as described with reference to FIGS. 11 to 13, machine learning with sufficiently high accuracy can be implemented. Alternatively, all points DP may be included in the targets of machine learning; because when all points DP are included in the targets of machine learning, model data D3 can be generated which enables more reliable classification of points DP as exclusion targets that do not correspond to bruxism.

The following describes the machine learning related to distinction of more detailed classifications in a case where bruxism is occurring with reference to FIG. 17.

FIG. 17 is a two-dimensional graph schematically illustrating machine learning related to classification of types of bruxism by a subject. For example, a point DP within a boundary SP1 and within a boundary SP11 illustrated in FIG. 17 corresponds to grinding among the types of bruxism. A point DP within the boundary SP1 and within a boundary SP12 corresponds to gnashing among the types of bruxism. A point DP within the boundary SP1 and within a boundary SP13 corresponds to tapping among the types of bruxism. A point DP within the boundary SP1 and not included in any of the boundaries SP11, SP12, and SP13 corresponds to clenching among the types of bruxism. Such classification of the types of bruxism can be implemented, for example, based on the parameter of “ground truth (type of bruxism)” among the parameters included in the distinction information AD2. The boundaries SP11, SP12 and SP13 are hyperplanes in the {(q×α)+β}-dimensional vector space, in the same manner as the boundary SP1.

Parameters other than “ground truth (type of bruxism)” can be taken into consideration for the classification of the types of bruxism. For example, a vector direction indicated by the intensity direction PW illustrated in FIG. 17 corresponds to the magnitude of the value of “ground truth (intensity)”. The boundary SP11 is therefore a classification in which the intensity of bruxism is relatively strong. The boundary SP13 is a classification in which the intensity of bruxism is relatively weak. The boundary SP12 is an intermediate classification in terms of the intensity of bruxism that can occur. This correlation between the intensity of bruxism and the classification of the types of bruxism can be further included in the learning related to the classification of the types of bruxism. Based on the same concept, a correlation can also be established between the classification of the type of bruxism and the “ground truth (location of bruxism)”, that is, the sensor producing an output corresponding to force among the plurality of sensors that function as the force sensor 20. This correlation between the location of bruxism and the classification of the types of bruxism can be further included in the learning related to the classification of the types of bruxism.

The training data D1 and the feature data D2 are not limited to data from a single subject. Machine learning may be performed based on data from a plurality of subjects, or learning that enables classification of points DP depending on subjects' tendency may be performed. For example, learning may be performed by regarding a plurality of subjects with identical or similar physical characteristics as “(a set of) subjects exhibiting similar tendencies” as the subjects' tendency. Information indicating the physical characteristics includes information on the subject's age, gender, height, weight, and the like.

FIG. 18 is a two-dimensional graph schematically illustrating machine learning related to classification of types of bruxism by a subject different from the subject in FIG. 17. The distribution of points DP illustrated in FIG. 18 differs from the distribution of points DP illustrated in FIG. 17. This is because the points DP illustrated in FIG. 18 are based on training data D1 and feature data D2 different from the training data D1 and the feature data D2 for the points DP illustrated in FIG. 17. The subject from whom the training data D1 for the points DP in FIG. 17 is obtained, is different from the subject from whom the training data D1 for the points DP in FIG. 18 is obtained. A boundary SP2 therefore represents a spatial region different from that of the boundary SP1. In FIG. 18, a point DP within the boundary SP2 is a point DP with a value of 1 for “ground truth (presence or absence of bruxism)”, that is, a point DP corresponding to a combination of force features and myoelectric features in a case where bruxism is occurring.

A point DP within the boundary SP2 and within a boundary SP21 illustrated in FIG. 18 corresponds to grinding among the types of bruxism. A point DP within the boundary SP2 and within a boundary SP22 corresponds to gnashing among the types of bruxism. A point DP within the boundary SP2 and within a boundary SP23 corresponds to tapping among the types of bruxism. A point DP within the boundary SP2 and not included in any of the boundaries SP21, SP22, and SP23 corresponds to clenching among the types of bruxism. The boundary SP2 and the boundaries SP21, SP22, and SP23 are hyperplanes in the {(q×α)+β}-dimensional vector space, in the same manner as the boundary SP1.

The learning processor 73 illustrated in FIG. 1 generates the model data D3 as an output of the supervised machine learning based on SVM, using the distinction information AD2 of the training data D1 (see FIG. 14) and the feature data D2 (see FIG. 15) as inputs (training data), as described with reference to FIG. 16 to FIG. 18. The model data D3 includes bruxism estimation model data 85 and bruxism detail identifying model data 86. The bruxism estimation model data 85 is model data that is used to determine data by which occurrence of bruxism is estimated. The bruxism detail identifying model data 86 is model data that is used to identify what the bruxism is like (type, location, intensity) in a case where occurrence of bruxism is estimated. The bruxism estimation model data 85 and the bruxism detail identifying model data 86 are referred to in the processing by the distinction processor 75.

The distinction processor 75 illustrated in FIG. 1 distinguish whether or not data acquired by the data acquisition processor 61 and having no distinction information AD2 added is the data by which occurrence of bruxism is estimated. When the occurrence of bruxism is estimated, the distinction processor 75 identifies what the bruxism is like (type, location, intensity). The data acquired by the data acquisition processor 61 and having no distinction information AD2 added is, for example, the distinction target data D4. Specifically, the distinction processor 75 functions, for example, when the arithmetic unit 70 executes the bruxism distinction program 76. The distinction processor 75 refers to the model data D3 using the distinction target data D4 as input data, and produces an output indicating the classification of the data based on SVM.

FIG. 19 is a diagram illustrating an example of the relation between the distinction target data D4 and distinction information OP indicating the output of the distinction processor 75 based on the distinction target data D4. The distinction target data D4 includes myoelectric data 87 and force data 88. The myoelectric data 87 is a column in which a parameter corresponding to the output of the myoelectric sensor 40 is registered, in the same manner as the myoelectric data 81. In the force data 88, a parameter corresponding to the output of the force sensor 20 is registered, in the same manner as in the force data 82. The force data 88 illustrated in FIG. 19 therefore includes three columns: “first force data”, “second force data”, and “third force data”, in the same manner as the force data 82 illustrated in FIG. 14. The distinction target data D4 is data that is acquired by the data acquisition processor 61 and to which the synchronization information AD1 described with reference to FIG. 14 is added, in the same manner as the training data D1. However, unlike the training data D1 described with reference to FIG. 14, the distinction target data D4 does not include the distinction information AD2. In other words, the distinction target data D4 is not training data but data to be distinguished for classification of data using model data based on machine learning.

The distinction processor 75 classifies each record of the distinction target data D4, according to the classifications of points DP by SVM with reference to the model data D3. Specifically, the distinction processor 75 generates first data as data similar to the myoelectric feature data 83 from the myoelectric data 87 of the distinction target data D4, and generates second data as data similar to the force feature data 84 from the force data 88, in the same manner that the feature acquisition processor 71 generates the feature data D2 from the training data D1. That is, the first data corresponds to the data denoted as “myoelectric feature” in the description with reference to FIGS. 16 to 18. The second data corresponds to the data denoted as “force feature” in the description with reference to FIGS. 16 to 18.

The distinction processor 75 regards a combination of myoelectric features and force features indicated by each record of the generated first and second data as a point DP, and plots the point DP in the {(q×α)+β}-dimensional vector space. The distinction processor 75 refers to the model data D3 and applies the boundaries indicating the classifications of points DP to the {(q×α)+β}-dimensional vector space. As used herein, the boundaries indicating the classifications of points DP are, for example, the boundaries SP1, SP11, SP12, and SP13 illustrated in FIG. 17 or boundaries SP2, SP21, SP22, and SP23 illustrated in FIG. 18. Based on the relation between the points DP and the boundaries indicating the classifications of points DP, the distinction processor 75 performs estimation of whether bruxism is occurring and identification of the details (type, location, intensity) of bruxism in a case where occurrence of bruxism is estimated.

In the embodiment, the distinction processor 75 adds, for example, the distinction information OP illustrated in FIG. 19 to the distinction target data D4, as the output indicating the results of estimation of whether bruxism is occurring and identification of details (type, location, intensity) of bruxism in a case where occurrence of bruxism is estimated. The distinction information OP includes four columns: “result (presence or absence of bruxism)”, “result (type of bruxism)”, “result (location of bruxism)”, and “result (intensity)”.

The parameter of “result (presence or absence of bruxism)” indicates the result of the estimation by the distinction processor 75 as to whether bruxism is occurring, based on the relation between the boundary SP1 or SP2 and the point DP. The rule for the parameter of “result (presence or absence of bruxism)” is similar to the rule for the parameter of the “ground truth (presence or absence of bruxism)”. Therefore, the parameter of “result (presence or absence of bruxism)” in a record corresponding to the point DP estimated as “bruxism is occurring” is set to “1”. The parameter of “result (presence or absence of bruxism)” in a record corresponding to the point DP estimated as “bruxism is not occurring” is set to “0”.

The parameter of “result (type of bruxism)” indicates the result of the identification by the distinction processor 75 as to the type of bruxism in a case where occurrence of bruxism is estimated, based on the relation between the boundaries SP1, SP11, SP12, and SP13 or the boundaries SP2, SP21, SP22, and SP23 and the points DP. The rule for the parameter of “result (type of bruxism)” is similar to the rule for the parameter of the “ground truth (type of bruxism)”.

The parameter of “result (location of bruxism)” indicates the result of the identification by the distinction processor 75 as to the sensor that produces an output indicating that force is being generated. The rule for the parameter of “result (location of bruxism)” is similar to the rule for the parameter of the “ground truth (location of bruxism)”.

The parameter of “result (intensity of bruxism)” indicates the result of the identification by the distinction processor 75 as to the intensity of bruxism in a case where occurrence of bruxism is estimated, based on the relation between the intensity direction PW (see FIGS. 17 and 18) and the points DP. The rule for the parameter of “result (intensity of bruxism)” is similar to the rule for the parameter of the “ground truth (intensity of bruxism)”.

The “estimation” in the expression “estimation of whether bruxism is occurring” is intended to indicate that it is a medical professional such as a medical doctor who diagnoses bruxism, not the detection system 100. In other words, the detection system 100 provides information including the distinction information OP to medical professionals as information, for example, to help diagnose whether a subject using the detection device 10 has bruxism. This information is provided, for example, via display output by the display 64. Medical professionals can use the information provided by the detection system 100 as an aid in making a diagnosis of bruxism for the subject. In this way, the expression “estimation” indicates that it is a medical professional who makes the final decision of diagnosis, and that the diagnosis is not completed and only in the estimation stage at the time of output of the distinction information OP by the detection system 100.

The rule is predetermined as to which of the boundary SP1 illustrated in FIG. 17 and the boundary SP2 illustrated in FIG. 18 is employed or as to whether model data D3 corresponding to other subjects is employed rather than the boundaries SP1 and SP2. For example, it is assumed that individual model data D3 is prepared in advance for each of a plurality of subjects. In this case, the following rule may be employed: the subject's personal information is associated with the model data D3 in advance, and the personal information is input via the operation device 63 when the estimation and identification is performed by the distinction processor 75. Thus, the model data D3 tailored to each subject is applied. A rule that associates the model data D3 with information indicating the subject's physical characteristics may be employed. The information indicating the physical characteristics includes information on the age, gender, height, weight, and the like of the subject from whom the training data D1 used in generating the model data D3 is obtained. Thus, the distinction processor 75 can perform the processing using model data D3 of a subject with similar physical characteristics, without preparing individual model data D3 for each subject.

In the above description of the processing by the distinction processor 75, the distinction processor 75 derives features in the same manner as the feature acquisition processor 71. However, the feature acquisition processor 71 may perform the processing of deriving features from the distinction target data D4, among the processing performed by the distinction processor 75. Even when the distinction processor 75 performs feature derivation, both the feature acquisition processor 71 and the distinction processor 75 may be able to refer to the filter 72.

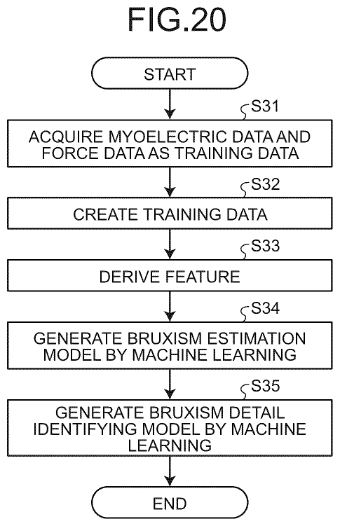

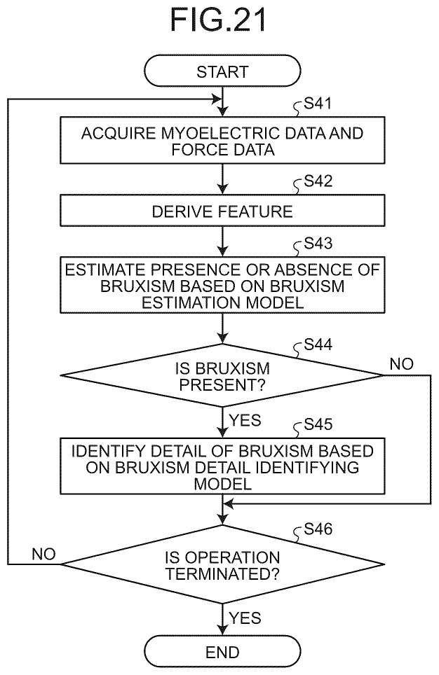

Among various matters pertaining to the detection system 100 described above, an overview of a process performed in the information processing device 60 will be described below with reference to FIGS. 20 and 21.

FIG. 20 is a flowchart illustrating an example of a process related to generation of model data D3. First, acquisition of myoelectric data and force data as training data is performed (step S31). Specifically, the training data D1 is acquired by the data acquisition processor 61. Next, creation of training data is performed (step S32). Specifically, the distinction information AD2 is added to the training data D1 acquired at step S31.

After the processing at step S32, feature derivation is performed (step S33). Specifically, the feature acquisition processor 71 derives the feature data D2, based on the myoelectric data 81 and the force data 82 of the training data D1 acquired at step S31 and having the distinction information AD2 added at step S32.

After the processing at step S33, generation of the bruxism estimation model data 85 by machine learning (step S34) and generation of the bruxism detail identifying model data 86 by machine learning (step S35) are performed. Specifically, the learning processor 73 performs machine learning as described with reference to FIGS. 16 to 18, based on the feature data D2 derived at step S33, and generates the model data D3, that is, the bruxism estimation model data 85 and the bruxism detail identifying model data 86. The processing at step S34 and the processing at step S35 are performed in no particular order.

FIG. 21 is a flowchart illustrating a process related to estimation of occurrence of bruxism and the like based on the distinction target data D4. First, acquisition of myoelectric data and force data is performed (step S41). Specifically, the data acquisition processor 61 acquires the distinction target data D4. Next, feature derivation is performed (step S42). Specifically, the distinction processor 75 or the feature acquisition processor 71 generates the first data and the second data, based on the myoelectric data 81 and the force data 82 of the distinction target data D4 acquired at step S41. The first data includes parameters similar to the first myoelectric feature AD4, the second myoelectric feature AD5, the third myoelectric feature AD6, and the fourth myoelectric feature AD7. The second data includes parameters similar to the first force feature AD8 and the second force feature AD9.

After the processing at step S42, estimation of the presence or absence of occurrence of bruxism is performed, based on the bruxism estimation model data 85 (step S43). Specifically, the distinction processor 75 regards a combination of myoelectric features and force features indicated by each record of the generated first and second data as a point DP, and plots the point in the {(q×α)+β}-dimensional vector space. The distinction processor 75 refers to the bruxism estimation model data 85 and applies a boundary related to the presence or absence of occurrence of bruxism (for example, boundary SP1 or boundary SP2), among the boundaries indicating the classifications of points DP, to the {(q×α)+β}-dimensional vector space. The distinction processor 75 performs estimation of whether bruxism is occurring, based on the relation between the points DP and the applied boundary. The result of the estimation in the processing at step S43 is reflected in the distinction information OP.

If it is estimated that bruxism is occurring in the processing at step S43 (Yes at step S44), identification of the details of bruxism is performed based on the bruxism detail identifying model data 86 (step S45). Specifically, the distinction processor 75 refers to the bruxism detail identifying model data 86 and applies boundaries related to the details of bruxism (for example, the boundaries SP1, SP11, SP12 and SP13 or the boundaries SP2, SP21, SP22 and SP23), among the boundaries indicating the classifications of the points DP, to the {(q×α)+β} dimensional vector space. The distinction processor 75 performs the identification of the details (type, location, intensity) of bruxism, based on the relation between the points DP and the applied boundaries in the processing at step S45. The result of the identification in the processing at step S45 is reflected in the distinction information OP.

After the processing at step S45 or when it is estimated that bruxism is not occurring in the processing at step S43 (No at step S44), the process moves to step S41 unless the operation of the information processing device 60 is terminated (No at step S46). If the operation of the information processing device 60 is terminated (Yes at step S46), the process related to the estimation of occurrence of bruxism and the like based on the distinction target data D4 ends.

With reference to FIGS. 14 to 21, the determination of items related to bruxism using machine learning has been described. However, machine learning is not essential for the determination of items related to bruxism, and the determination may be rule-based. For example, as described above with reference to FIGS. 9 and 10, the presence or absence of bruxism can be sufficiently determined, based on the synchronization of the output of the force sensor 20 and the output of the myoelectric sensor 40, and the relation between these outputs and the thresholds (for example, the first threshold Th1, the second threshold Th2).

According to the embodiment described above, the detection system 100 includes a detection device (for example, detection device 10) configured to perform sensing related to bruxism, and an information processing device (for example, information processing device 60) configured to determine the presence or absence of bruxism based on data indicating the result of sensing by the detection device. The detection device includes a force sensor (for example, first sensor element 221, second sensor element 222, third sensor element 223) disposed at a mouthpiece (for example, mouthpiece 21), a myoelectric sensor (for example, myoelectric sensor 40) attachable to a human cheek, and a controller (for example, controller 12) configured to output data in which an output of the force sensor is synchronized with an output of the myoelectric sensor (for example, myoelectric data 81 and force data 82 in the training data D1, myoelectric data 87 and force data 88 in the distinction target data D4). This configuration can present a relation between the output of the force sensor and the output of the myoelectric sensor at each point in time during a period of time in which the output of the force sensor and the output of the myoelectric sensor are produced. Therefore, compared with the case only using the force sensor or the case only using the myoelectric sensor, data caused by bruxism and data not caused by bruxism can be distinguished from each other more accurately.

The information processing device (for example, information processing device 60) includes a storage (for example, storage 80) configured to store feature data (for example, feature data D2) and model data (for example, model data D3). The feature data is data in which the myoelectric feature (for example, myoelectric feature data 83) corresponding to the output of the myoelectric sensor (for example, myoelectric sensor 40) is synchronized in time with the force feature (for example, force feature data 84) corresponding to the output of the force sensor (for example, first sensor element 221, second sensor element 222, third sensor element 223). The model data is data indicating a correlation between a combination of the myoelectric feature and the force feature and an item related to bruxism. The myoelectric feature includes data obtained by filtering the output of the myoelectric sensor to a specific frequency component (for example, M₃ (ω)=H₃ (ω) M (w), M₄ (ω)=H₄ (ω) M (ω) described above). The force feature includes data obtained by filtering the output of the force sensor to a specific frequency component (for example, P₂ (ω)=H₆ (ω) P₁ (ω) described above). With this configuration, data can be classified using a frequency component that is included in the data and can be used more suitably for data classification, compared with when data is classified based on sensing data output as it is. As a result, it is possible to more accurately distinguish between data caused by bruxism and data not caused by bruxism.

The item related to bruxism in the model data (for example, model data D3) includes the type of bruxism. The information processing device (for example, information processing device 60) includes a learning processor (for example, learning processor 73) configured to generate the model data, based on feature data (for example, feature data D2) and training data (for example, training data D1) including information indicating the type of bruxism (for example, distinction information AD2). With this configuration, the information processing device can generate the model data that reflects a correlation between the feature data and the information indicating the type of bruxism.

The myoelectric feature (for example, myoelectric feature data 83) includes data obtained by filtering the output of the myoelectric sensor (for example, myoelectric sensor 40) with a bandpass filter. With this configuration, it is possible, by an easier method of limiting a frequency component by filtering, to obtain a frequency component that can be used more suitably for data classification.

Model data (for example, model data D3) for each of a plurality of humans (for example, see FIGS. 17 and 18) who use the detection device (for example, detection device 10) is stored in the storage (for example, storage 80). The information processing device (for example, information processing device 60) determines the presence or absence of bruxism based on the model data corresponding to the human who uses the detection device, when determining the presence or absence of bruxism based on new data (for example, distinction target data D4). This configuration enables determination based on the model data more appropriate for the human who uses the detection device. As a result, it is possible to more accurately distinguish between data caused by bruxism and data not caused by bruxism.

A plurality of force sensors (for example, first sensor element 221, second sensor element 222, third sensor element 223) are disposed at the mouthpiece (for example, mouthpiece 21), and the controller (for example, controller 12) outputs data in which outputs of the force sensors can be individually identified (see, for example, FIG. 9). With this configuration, the position in the human mouth where the force is generated can be located more accurately, based on the relation between the arrangement of each of force sensors assumed in the human mouth (see, for example, FIG. 6) and the output of each of the force sensors in the data.

The force sensor (for example, first sensor element 221, second sensor element 222, third sensor element 223) is provided at a flexible substrate (for example, FPC 25), and the flexible substrate is mounted on the mouthpiece (for example, mouthpiece 21). With this configuration, the output of the force sensor can be transmitted via the flexible substrate. The flexibility of the flexible substrate makes it easier to achieve both flexibility for human mouth movement and a configuration for more reliable transmission of output of the force sensor.

The flexible substrate (for example, FPC 25) is adhesively fixed such that a gap is formed between the flexible substrate and the mouthpiece, inside the mouthpiece (for example, mouthpiece 21) (see, for example, FIG. 4). This configuration easily prevents or reduces the force on the mouthpiece from acting directly on the flexible substrate. This configuration also more easily enhances flexibility and engagement of the mouthpiece with protrusions and depressions of the teeth in the human mouth.

The myoelectric sensor (for example, myoelectric sensor 40) includes an electrode (for example, first electrode 42, second electrode 43, and third electrode 44), and gel (for example, conductive gels 42a, 43a, 44a) and double-sided tape (for example, double-sided tape 45 for skin) are applied on an attachment surface of the myoelectric sensor that is provided with the electrode. This configuration facilitates attachment of the myoelectric sensor to the human while further ensuring that the electrode is in close proximity to the human cheek.

The controller (for example, controller 12) causes the myoelectric sensor (for example, myoelectric sensor 40) to operate at a first cycle until the output of the myoelectric sensor exceeds a first threshold (for example, first threshold Th1), and causes the myoelectric sensor to operate at a second cycle after the output of the myoelectric sensor exceeds the first threshold. The second cycle is a higher-frequency cycle than the first cycle. As used herein, the first cycle is, for example, the low-frequency cycle in the description with reference to FIG. 11. As used herein, the second cycle is, for example, the high-frequency cycle in the description with reference to FIG. 11. This configuration gives higher priority to power saving until the output of the myoelectric sensor that may be an output corresponding to bruxism is produced. After the output of the myoelectric sensor that may be an output corresponding to bruxism is produced, more accurate sensing can be performed with more frequent outputs.

The controller (for example, controller 12) causes the force sensor (for example, force sensor 20) to operate at a third cycle until the output of the force sensor exceeds a second threshold (for example, second threshold Th2), and causes the force sensor to operate at a fourth cycle after the output of the force sensor exceeds the second threshold. The fourth cycle is a higher-frequency cycle than the third cycle. As used herein, the third cycle is, for example, the low-frequency cycle in the description with reference to FIG. 12. As used herein, the fourth cycle is, for example, the high-frequency cycle in the description with reference to FIG. 12. This configuration gives higher priority to power saving until the output of the force sensor that may be an output corresponding to bruxism is produced. After the output of the force sensor that may be an output corresponding to bruxism is produced, more accurate sensing can be performed with more frequent outputs.

The detection device 10 further includes a communication circuit (for example, communication circuit 13) that communicates with an external apparatus (for example, information processing device 60). The controller (for example, controller 12) does not cause the communication circuit to operate when neither a first condition nor a second condition is satisfied, and causes the communication circuit to operate when at least one of the first condition and the second condition is satisfied. The first condition is that the output of the myoelectric sensor (for example, myoelectric sensor 40) exceeds a first threshold (for example, first threshold Th1). The second condition is that the output of the force sensor (for example, force sensor 20) exceeds a second threshold (for example, second threshold Th2). This configuration gives higher priority to power saving until the output of at least one of the myoelectric sensor and the force sensor that may be an output corresponding to bruxism is produced. After an output that may be an output corresponding to bruxism is produced, more accurate sensing can be performed with outputs at a higher frequency.

In the foregoing embodiment, the learning processor 73 generates the model data D3 as the output of supervised machine learning based on SVM using the distinction information AD2 of the training data D1 and the feature data D2 as inputs (training data). However, the embodiment is not limited to this. For example, the distinction information AD2 may be copied from the training data D1 to the feature data D2. In this case, the learning processor 73 can perform supervised machine learning by simply referring to the feature data D2.

Features obtained by changing the filter to a different filter may be additionally included as parameters included in the myoelectric feature data 83 and the force feature data 84 in the feature data D2. For example, features corresponding to a median frequency (MDF), a mean frequency (MNF), or the like may be added to the myoelectric feature data 83. Features with a different time average width and/or features to which a low-pass filter, a high-pass filter, or a band-pass filter is applied may be included in the force feature data 84.

The algorithm for generating the model data D3 is not limited to SVM. In particular, the bruxism detail identifying model data 86 may be generated using other algorithms such as decision trees and logistic regression.

The specific configuration of the force sensor 20 is not limited to the one using the piezoelectric effect described above. For example, the specific configuration of the sensor element 22 may be a strain gauge, and a circuit including a Wheatstone bridge may be provided in the casing 41. In this case, strain generated in each of the first sensor element 221, the second sensor element 222, and the third sensor element 223 produces an output representing force. A plurality of sensor elements, such as the first sensor element 221, the second sensor element 222, and the third sensor element 223, are not necessarily provided, and a sensor element in a curved shape along the shape of human teeth may be employed. In addition, the specific configuration of the force sensor 20 may be a resistive force sensor. However, the force sensor 20 is preferably a film-type force sensor.

Other effects brought about by the manners described in the present embodiment that are obvious from the description here or that can be conceived by a person skilled in the art should be understood to be brought about by the present disclosure.

What is claimed is:

1. A detection system comprising:
a detection device configured to perform sensing related to bruxism; and
an information processing device configured to determine presence or absence of bruxism based on data indicating a result of sensing by the detection device, wherein
the detection device includes
a force sensor disposed at a mouthpiece,
a myoelectric sensor attachable to a human cheek, and
a controller configured to output data in which an output of the force sensor is synchronized with an output of the myoelectric sensor.

2. A detection system comprising:
a detection device configured to perform sensing related to bruxism; and
an information processing device configured to determine presence or absence of bruxism based on data indicating a result of sensing by the detection device, wherein
the detection device includes
a mouthpiece having a force sensor,
a myoelectric sensor attachable to a human cheek, and
a controller configured to synchronize an output of the force sensor with an output of the myoelectric sensor and output the synchronized outputs as the data,
the information processing device includes
a storage configured to store feature data and model data,
the feature data is data in which a myoelectric feature corresponding to the output of the myoelectric sensor is synchronized in time with a force feature corresponding to the output of the force sensor,
the model data is data indicating a correlation between a combination of the myoelectric feature and the force feature and an item related to bruxism,
the myoelectric feature includes data obtained by filtering the output of the myoelectric sensor to a specific frequency component, and
the force feature includes data obtained by filtering the output of the force sensor to a specific frequency component.

3. The detection system according to claim 2, wherein
the item related to bruxism includes a type of bruxism, and
the information processing device includes a learning processor configured to generate the model data based on the feature data and training data including information indicating a type of bruxism.

4. The detection system according to claim 2, wherein the myoelectric feature includes data obtained by filtering the output of the myoelectric sensor with a bandpass filter.

5. The detection system according to claim 2, wherein
the model data for each of a plurality of humans who use the detection device is stored in the storage, and
the information processing device configured to determine presence or absence of bruxism based on the model data corresponding to a human who uses the detection device, when determining the presence or absence of bruxism based on new data indicating a result of sensing.
